(19) United States
(12) Patent Application Publication
(10) Pub. No.: US 2022/0202373 A1
(43) Pub. Date: Jun. 30, 2022

(54) SYSTEMS AND METHODS OF USING MACHINE LEARNING TO DETECT AND PREDICT EMERGENCE OF AGITATION BASED ON SYMPATHETIC NERVOUS SYSTEM ACTIVITIES

(71) Applicant: BioXcel Therapeutics, Inc., New Haven, CT (US)

(72) Inventors: Frank D. YOCCA, New Haven, CT (US); Michael De Vivo, New Haven, CT (US); Robert Risinger, New Haven, CT (US); Subhendu Seth, Gurgaon (IN); Martin Majernik, Bratislava (SK); Daniel R. Karlin, New York, NY (US); Jamileh Jemison, Waltham, MA (US); Alexander Wald, Bratislava (SK); Miguel Amável Dos Santos Pinheiro, Praha (CZ)

(73) Assignee: BioXcel Therapeutics, Inc., New Haven, CT (US)

(21) Appl. No.: 17/698,407

(22) Filed: Mar. 18, 2022

Related U.S. Application Data
(63) Continuation of application No. PCT/US2020/051256, filed on Sep. 17, 2020.
(60) Provisional application No. 62/976,685, filed on Feb. 14, 2020.
(60) Provisional application No. 62/901,955, filed on Sep. 18, 2019.

(51) Int. Cl. A61B 5/00; A61B 5/372; G06F 3/01; G06N 20/00

(52) U.S. Cl. CPC A61B 5/7264; A61B 5/7275; A61B 5/7282; A61B 5/746; G06N 20/00; A61B 5/0006; A61B 5/372; G06F 3/015; A61B 5/7465

(57) ABSTRACT

In some embodiments, a method includes receiving first physiological data of sympathetic nervous system activity and establishing a baseline value of at least one physiological parameter by training at least one machine learning model using the first physiological data. The method further includes receiving, from a first monitoring device attached to a subject, second physiological data of sympathetic nervous system activity in the subject. Using the at least one machine learning model and based on the baseline value of at least one physiological parameter, the method includes analyzing the second physiological data to predict an agitation episode of the subject and sending a signal to a second monitoring device to notify of the prediction of the agitation episode of the subject such that treatment can be provided to the subject to decrease sympathetic nervous system activity in the subject.

### CROSS-REFERENCE TO RELATED APPLICATIONS

This application is a Continuation of International Patent Application No. PCT/US2020/051256, filed Sep. 17, 2020, which claims priority to and benefit of U.S. Provisional Application No. 62/976,685, filed Feb. 14, 2020, and U.S. Provisional Application No. 62/901,955, filed Sep. 18, 2019, the entire disclosure of each of which is incorporated herein by reference in its entirety.

### FIELD

The present disclosure provides a method of using machine learning to detect and predict an agitation event of a subject based on sympathetic nervous system activities, and treating said subject with an anti-agitation agent prior to the emergence of agitation.

### BACKGROUND

Agitation is characterized by excessive motor or verbal activity, irritability, uncooperativeness, threatening gestures, and, in some cases, aggressive or violent behavior. Subjects with schizophrenia are particularly vulnerable to acute episodes of agitation, especially during exacerbation of the disease. Agitation associated with psychosis is also a frequent reason for emergency department visits, and unless recognized early and managed effectively, can rapidly escalate to a potentially dangerous situation, including physical violence. Agitation is not a specific disorder, but it is a common sign or symptom in many acute and chronic neurological or psychiatric conditions. Thought to be a response to an underlying disturbance or trigger, agitation may manifest as restlessness, wandering, pacing, fidgeting, rapid speech or verbal outbursts among other signs of hyperarousal. Agitation is frequently disruptive and in some people may escalate to acts of aggression. For this reason, it is a symptom that can lead to institutionalization of individuals who might otherwise be able to be cared for at home, and diminishes the quality of life of subjects and caregivers. Tracking of agitation behavior and characterization of patterns in an individual's agitated state could reveal signals of agitation onset, allowing earlier efforts to de-escalate, and reducing the need for medical intervention, sedating medications, or restraint.

Unfortunately, clinicians do not always diagnose episodes of agitation early enough to prevent such an escalation. Therefore, a need exists for (1) a tool to measure the signs of an impending agitation event, and alert the caregiver to treat the subject before the emergence of agitation and (2) a suitable treatment, which may include the administration of an anti-agitation agent, to calm the subject and prevent an agitation episode from occurring. These and related desiderata have been met by the present disclosure.

### SUMMARY

The following disclosure presents a simplified summary of the disclosure in order to provide a basic understanding of some aspects of the disclosure. This summary is not an extensive overview of the present disclosure. It is not intended to identify the key/critical elements of the disclosure or to delineate the scope of the disclosure. Its sole purpose is to present some concept of the disclosure in a simplified form as a prelude to a more detailed description of the disclosure presented later.

In some embodiments, a method includes receiving first physiological data of sympathetic nervous system activity and establishing a baseline value of at least one physiological parameter by training at least one machine learning model using the first physiological data. The method further includes receiving, from a first monitoring device attached to a subject, second physiological data of sympathetic nervous system activity in the subject. Using the at least one machine learning model and based on the baseline value of at least one physiological parameter, the method includes analyzing the second physiological data to predict an agitation episode of the subject and sending a signal to a second monitoring device to notify of the prediction of the agitation episode of the subject such that treatment can be provided to the subject to decrease sympathetic nervous system activity in the subject.

An object of the present disclosure is to provide a solution for diagnosing an impending agitation episode in a subject predisposed to agitation.

Another object of the present disclosure is to provide a solution for alerting a caregiver to an impending agitation episode in a subject predisposed to agitation.

Yet another object of the present disclosure is to provide a solution for treating the early stage emergence of agitation or the signs of agitation in a subject predisposed to agitation.

The present disclosure provides an integrated system for preventing the emergence of agitation, comprising (A) an automated device which both monitors sympathetic nervous system activity (for example by measuring changes in electrodermal activity (EDA), heart rate variability, pupil size, secretion of salivary amylase, muscle activity, body temperature, motor activities, audio signals etc.) in a subject predisposed to agitation, and alerts a caregiver to an impending agitation episode, and (B) a treatment component where the subject identified with emerging agitation is administered an anti-agitation agent to prevent the manifestation of an agitation episode.

The present disclosure also describes a method to detect physiological measures of cardiovascular and motor activity that reliably predict emergence of agitation within a few hours, e.g. about 2 hours or less.

Thus, in a first aspect, the present disclosure provides a method of diagnosing an impending agitation episode in a subject predisposed to agitation comprising:

(a) monitoring one or more physiological signals of sympathetic nervous system activity in the subject using an automated sensoring device placed or mounted on the subject's skin surface; and
(b) identifying, via the processing of incoming data in the device, when the subject is about to have an agitation episode.

In a second aspect, the present disclosure provides a method of alerting a caregiver to an impending agitation episode in a subject predisposed to agitation comprising:
(a) monitoring one or more physiological signals of sympathetic nervous system activity in the subject using an automated sensoring device placed or mounted on the subject's skin surface;
(b) identifying, via the processing of incoming data in the device, when the subject is about to have an agitation episode; and
(c) sending a signal from the device to a compatible device monitored by a caregiver alerting the caregiver to an impending agitation episode in the subject.

In a third aspect, the present disclosure provides a method of preventing the emergence of agitation in a subject predisposed to agitation comprising:
(a) monitoring one or more physiological signals of sympathetic nervous system activity in the subject using an automated sensoring device placed or mounted on the subject's skin surface;
(b) identifying, via the processing of incoming data in the device, when the subject is about to have an agitation episode;
(c) sending a signal from the device to a remote compatible device monitored by a caregiver alerting the caregiver to an impending agitation episode in the subject; and
(d) administering by a caregiver an anti-agitation agent which decreases sympathetic nervous activity in said subject.

In a fourth aspect, the present disclosure provides a method of treating the early stage emergence of agitation or the signs of agitation in a subject predisposed to agitation comprising:
(a) monitoring one or more physiological signals of sympathetic nervous system activity in the subject using an automated sensoring device placed or mounted on the subject's skin surface;
(b) identifying, via the processing of incoming data in the device, when the subject is having an agitation episode;
(c) sending a signal from the device to a remote compatible device monitored by a caregiver alerting the caregiver to the start of agitation episode in the subject; and
(d) administering by the caregiver an anti-agitation agent which decreases sympathetic nervous activity in said subject.

In a fifth aspect, the present disclosure provides a method of preventing the emergence of agitation in a subject predisposed to agitation without causing significant sedation comprising:
(a) monitoring one or more physiological signals of sympathetic nervous system activity in the subject using an automated sensoring device placed or mounted on the subject's skin surface;
(b) identifying, via the processing of incoming data in the device, when the subject is about to have an agitation episode;
(c) sending a signal from the device to a remote compatible device monitored by a caregiver alerting the caregiver to an impending agitation episode in the subject; and
(d) administering by the caregiver an anti-agitation agent which decreases sympathetic nervous activity in said subject without causing significant sedation.

In a sixth aspect, the present disclosure provides a method of treating the early stage emergence of agitation or the signs of agitation in a subject predisposed to agitation without causing significant sedation comprising:
(a) monitoring one or more physiological signals of sympathetic nervous system activity in the subject using an automated sensoring device placed or mounted on the subject's skin surface;
(b) identifying, via the processing of incoming data in the device, when the subject is having an agitation episode;
(c) sending a signal from the device to a remote compatible device monitored by a caregiver alerting the caregiver to the start of agitation episode in the subject; and
(d) administering by the caregiver an anti-agitation agent which decreases sympathetic nervous activity in said subject without causing significant sedation.

In a seventh aspect, the present disclosure provides a method, comprising:
(a) receiving first physiological data of sympathetic nervous system activity;
(b) establishing a baseline value of at least one physiological parameter by training at least one machine learning model using the first physiological data;
(c) receiving, from a first monitoring device attached to a subject, second physiological data of sympathetic nervous system activity in the subject;
(d) analyzing, using the at least one machine learning model and based on the baseline value of at least one physiological parameter, the second physiological data to predict an agitation episode in the subject; and
(e) sending, based on predicting the agitation episode of the subject, a signal to a second monitoring device to notify the second monitoring device of the prediction of the agitation episode in the subject such that treatment can be provided to the subject to decrease sympathetic nervous system activity in the subject.

In an eighth aspect, the present disclosure provides a system for determining the emergence of agitation or the signs of agitation in a subject predisposed to agitation, comprising:
(a) an automated sensoring device configured to monitor at least sympathetic nervous system activity in the subject predisposed to agitation;
(b) a data collection unit configured to passively collect data from at least the wearable device; wherein the data collection module is configured to communicate the data to a local server and to a network server; and
(c) a processing unit configured to conduct an Ecological Momentary Assessment (EMA) and to generate a report;
(d) wherein the processing unit is configured to diagnose an impending agitation episode in the subject and to send a signal to a compatible device monitored by a caregiver alerting the caregiver about an impending agitation episode in the subject.

In a ninth aspect, the present disclosure provides an apparatus, comprising: a memory; and a processor operatively coupled to the memory, the processor configured to: receive, from a first monitoring device attached to a subject, physiological data of sympathetic nervous system activity in the subject; analyze, using at least one machine learning model, the physiological data to detect an anomaly from a reference pattern of sympathetic nervous system activity to determine a probability of an occurrence of an agitation episode of the subject; and send a signal to a second monitoring device to notify the second monitoring device of the probability of the occurrence of the agitation episode of the subject such that treatment can be provided to the subject to decrease sympathetic nervous system activity in the subject. In some embodiments, the monitoring devices also detects the severity of the agitation (e.g., mild, moderate or elevated). In some embodiments, the monitoring device predicts the probability of specific patient to move from mild to moderate to elevated agitation.

In a tenth aspect, the present disclosure provides a processor-readable non-transitory medium storing code representing instructions to be executed by a processor, the code comprising code to cause the processor to: receive, from a first monitoring device attached to a subject, physiological data of sympathetic nervous system activity in the subject; analyze, using at least one machine learning model, the physiological data to detect an anomaly from a reference pattern of sympathetic nervous system activity to determine a probability of an occurrence of an agitation episode in the subject; and send a signal to a second monitoring device to notify the second monitoring device of the probability of the occurrence of the agitation episode of the subject such that treatment can be provided to the subject to decrease sympathetic nervous system activity in the subject.

Other salient features and advantages of the disclosure will become apparent to those skilled in the art from the following detailed description, which, taken in conjunction with the annexed drawings, discloses exemplary embodiments of the disclosure.

### BRIEF DESCRIPTION OF THE DRAWINGS

The above and other aspects, features, and advantages of certain example embodiments of the present disclosure will be more apparent from the following description taken in conjunction with the accompanying drawings in which:

FIG. 1 illustrates a system for determining the emergence of agitation or the signs of agitation in a subject predisposed to agitation according to an embodiment of the present disclosure.

FIG. 2 illustrates an ETL process overview for the disclosed system according to an embodiment of the present disclosure.

FIG. 3 illustrates a block diagram of a method of diagnosing an impending agitation episode in a subject predisposed to agitation according to an embodiment of the present disclosure.

FIG. 4 illustrates a block diagram of a method of alerting a caregiver to an impending agitation episode in a subject predisposed to agitation according to an embodiment of the present disclosure.

FIG. 5 illustrates a block diagram of a method of preventing the emergence of agitation in a subject predisposed to agitation according to an embodiment of the present disclosure.

FIG. 6 illustrates a block diagram of a method of treating the early stage emergence of agitation or the signs of agitation in a subject predisposed to agitation according to an embodiment of the present disclosure.

FIG. 7 illustrates a block diagram of method of diagnosing an impending agitation episode in a subject predisposed to agitation and alerting a caregiver according to another embodiment of the present disclosure.

FIG. 8 illustrates a block diagram of an apparatus to receive data, to analyze, using at least one machine learning model, and to send a signal to caregiver according to another embodiment of the present disclosure.

FIG. 9 illustrates a system flow diagram of a process to assign Patient IDs, Patient registration and recording of the data according to another embodiment of the present disclosure.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various example embodiments of the present disclosure. Throughout the drawings, it should be noted that like reference numbers are used to depict the same or similar elements, features, and structures.

### DETAILED DESCRIPTION

The following description with reference to the accompanying drawings is provided to assist in a comprehensive understanding of exemplary embodiments of the disclosure. It includes various specific details to assist in that understanding but these are to be regarded as merely examples.

Accordingly, a person skilled in the art will recognize that various changes and modifications of the embodiments described herein can be made without departing from the scope of the disclosure. In addition, descriptions of well-known functions and constructions are omitted for clarity and conciseness.

The terms and words used in the following description are not limited to the bibliographical meanings, but, are merely used by the inventor to enable a clear and consistent understanding of the disclosure. Accordingly, it should be apparent to those skilled in the art that the following description of exemplary embodiments of the present disclosure are provided for illustration purpose only and not for the purpose of limiting the disclosure as defined by their equivalents.

It is to be understood that the singular forms “a”, “an,” and “the” include plural referents unless the context clearly dictates otherwise.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, integers, steps or components but does not preclude the presence or addition of one or more other features, integers, steps, components or groups thereof

Abbreviations

ACES: Agitation and Calm Evaluation Scale

EDA: Electrodermal Activity

EEG: Electroencephalography

ETL: Extract, Transform and Load

EMA: Ecological Momentary AssessmentGLONASS: GLObal NAvigation Satellite System

HEOG: Horizontal Electrooculogram

VEOG: Vertical Electrooculogram

RASS: Richmond Agitation Sedation ScaleNavIC: Navigation with Indian Constellation

OPD: Out-patient Department

PC: Personal computer

PSG: Polysomnogram RHR: resting heart rate

IPD: In-patient Department

ICU: Intensive Care Unit

MMSE: Mini Mental State Exam

UP: Unanticipated Problems

In some embodiments, the terms “subject” and “patient” are used interchangeably herein, and mean any animal, including mammals, such as mice, rats, other rodents, rabbits, dogs, cats, swine, cattle, sheep, horses, or primates, such as humans.

In some embodiments, the term “subject predisposed to agitation” non-limitedly includes a subject with post-traumatic stress disorder, a neuropsychiatric condition/disease or a neurodegenerative condition/disease, a subject suffering from opioid, alcohol or substance abuse withdrawal (including cocaine, amphetamine), or a subject undergoing an OPD/IPD procedure.

In some embodiments, the term “dosage” non-limitedly is intended to encompass a formulation expressed in terms of μg per day, μg/kg, μg/kg/hr, μg/kg/day, mg/kg/day, or mg/kg/hr.

In some embodiments, a “dose” is an amount of an agent administered to a patient in a unit volume or mass, e.g., an absolute unit dose expressed in mg of the agent. The dose depends on the concentration of the agent in the formulation, e.g., in moles per litre (M), mass per volume (m/v), or mass per mass (m/m).

In some embodiments, the term “sedation” as used herein means depressed consciousness in which a patient or subject retains the ability to independently and continuously maintain an open airway and a regular breathing pattern, and to respond appropriately and rationally to physical stimulation and verbal commands. As used herein “without causing significant sedation” means that the patient experiences a level of sedation not greater than Level 3 on the Ramsay Sedation Scale. Level 3 means sedated but responds to commands.

In some embodiments, the term “emergence of agitation” as used herein refers to patients who are on the verge getting agitated, but the patient's body does not yet show signs of agitation via relevant mental and/or physical changes. If monitored properly, physiological signals may be used to measure sympathetic nervous activity and therefore can become markers of the emergence of the agitation. The present disclosure thus provides the monitoring of the emergence of agitation by identifying increased sympathetic nervous system activity from physiological signals such as changes in Electrodermal activity (skin conductance response) and changes in resting EEG.

In some embodiments, the term “the signs of agitation” non-limitedly as used herein includes excessive motor activity (examples include: pacing, rocking, gesturing, pointing fingers, restlessness, performing repetitious mannerisms), verbal aggression (e.g. yelling, speaking in an excessively loud voice, using profanity, screaming, shouting, threatening other people), physical aggression (e.g. grabbing, shoving, pushing, clenching hands into fists, resisting, hitting others, kicking objects or people, scratching, biting, throwing objects, hitting self, slamming doors, tearing things, and destroying property).

In some embodiments, the term “agitation”, non-limitedly as used herein, means irritability, emotional outburst, impaired thinking, or excess motor and verbal activity that may occur due to either dysfunction of specific brain regions such as frontal lobes or due to dysfunction of neurotransmitter systems such as dopamine and nor-epinephrine. In the present disclosure, agitation also includes aggression and hyper-arousal in post-traumatic stress disorder. The agitation may be acute or chronic. An occurrence of “agitation” is referred to herein as an “agitation episode” or an “agitation event”.

In some embodiments, the term “neuropsychiatric conditions/disease” as used herein includes, but is not limited to, schizophrenia, bipolar illness (bipolar disorder, bipolar mania), depression, major depressive disorder, delirium or other related neuropsychiatric conditions.

In some embodiments, the term “neurodegenerative conditions/disease” as used herein includes, but is not limited to, Alzheimer's disease, frontotemporal dementia (FTD), dementia, dementia with Lewy bodies (DLB), post-traumatic stress disorder, Parkinson's disease, vascular dementia, vascular cognitive impairment, Huntington's disease, multiple sclerosis, creutzfeldt-Jakob disease, multiple system atrophy, progressive supranuclear palsy, traumatic brain injury and or other related neurodegenerative diseases.

In some embodiments, the term “sublingual” literally means “under the tongue” and refers to a method of administering substances via the mouth in such a way that the substances are rapidly absorbed via the blood vessels under the tongue rather than via the digestive tract. Sublingual absorption occurs through the highly vascularized sublingual mucosa, which allows a substance direct access to the blood circulation, thereby providing for direct systemic administration independent of gastrointestinal influences and avoiding undesirable first-pass hepatic metabolism.

In some embodiments, the term “EDA”, as used herein, refers to electrodermal activity/response, which is also known as skin conductance response (and in older terminology as “galvanic skin response”). EDA is the phenomenon where the skin momentarily becomes a better conductor of electricity when either external or internal stimuli occur that are physiologically arousing. EDA is considered one of the fastest-responding physiological measures of stress response and arousal. The study of EDA has led to important tools such as EEG. An automated sensoring device placed on the skin of the patient, monitors the EDA by recording the changes in the patient's skin's electrical resistance. Any change in sympathetic nervous system activity results in a slight increase in perspiration, which lowers skin resistance (because perspiration contains water and electrolytes). Such changes in the skin's electrical resistance are recorded by the sensoring device.

In some embodiments, the term “EEG”, as used herein, refers to electroencephalography (EEG). EEG is an electrophysiological monitoring method to record electrical activity of the brain. EEG reflects the electrical activity of the underlying neurons, and provides information regarding neuronal population oscillations, the information flow pathway, and neural activity networks.

In some embodiments, the term “resting EEG”, as used herein, refers to EEG recordings taken in a resting state and denotes spontaneous neural activity, which is relevant to the fundamental brain state. Appropriate features derived from resting EEG may be helpful in monitoring the brain conditions of patients suffering from neuropsychiatric disease, neurodegenerative disease and other nervous system related disease. Resting EEG can therefore contribute to decision-making related to the care of such patients.

In some embodiments, the term “RASS” refers to the Richmond Agitation Sedation Scale: Change from baseline: The RASS is a 10-level rating scale ranging from “Combative” (+4) to “unarousable” (−5).

In some embodiments, the term “heart rate variability” refers to the variability of the time interval between heartbeats and is a reflection of an individual's current health status.

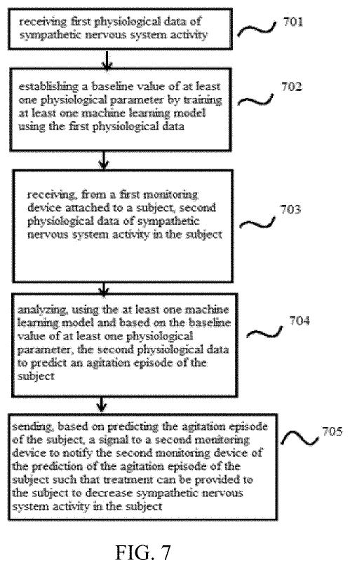

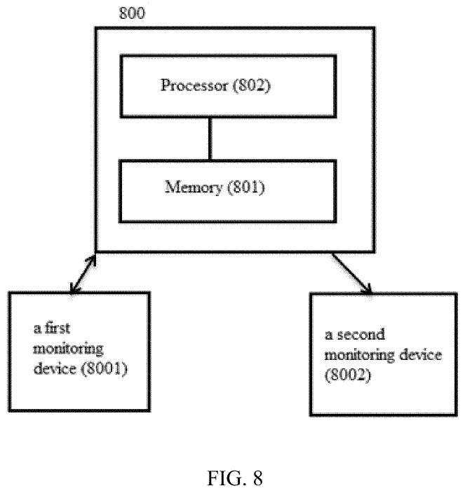

In some embodiments, the term “automated monitoring device” is used herein interchangeably with “automated sensoring device” and refers to any device that could be worn/placed/mounted on the body of the patient and that is able to detect, and process signals related to sympathetic nervous system activity and/or motor activity. The automated monitoring device is also referred to as “the first monitoring device” described with regards to FIG. 7 and FIG. 8. The device may interact (e.g., remotely or otherwise) with any suitable compatible device, such as an end-user display terminal, and will normally include transducers, a transducer control module, a communications device, and a monitoring system or a computer database etc. Physiological measures can also be measured using both standard technology and miniaturized wearable devices such as, for example, sensor devices (e.g., waist worn, wrist worn, finger worn, etc.) with networking capacity (e.g., an iPhone). The automated sensoring device used herein, collects the data on integrated physiological parameters (such as EDA, resting EEG, blood pressure, mobility/motor, memory/processing, speech/sleep patterns etc.) and then transfer/signal the collected data to a computer database external to the patient monitoring device including one or more early warning unit based on an early warning algorithm to transform data into a format that is interpretable as a specific measure, or, an aggregate functional outcome in the form of alert signals. The present disclosure provides an integrated patient management solution, which may enable early intervention for agitation via an analytic algorithm that predicts and identifies agitation. The automated sensoring device used herein can measure minimally observable changes in sympathetic nervous system activity of patients to a higher level of resolution than possible by clinical observation.

The automated monitoring device is capable of signaling information related to increases in sympathetic nervous system activity and motor activity to an apparatus (for example, a computer database) that is monitored by, for example, a caregiver. The automated monitoring device, for example, can be any suitable sensor device such as, for example, a waist worn multi-sensor device with networking capability, a wrist worn multi-sensor device with networking capability, a finger worn multi-sensor device with networking capability, and/or the like. A wide range of devices/sensors, such as, for example, a smartphone (e.g., iPhone (BYOD or provisioned)), accelerometers and gyroscopes, portable devices, digital devices, smart fabrics, bands and actuators, smartwatch (e.g., an Apple watch (e.g., Apple watch 3) or iWatch), patch such as MC10 Patch, Oura rings (for example, for patients unable to or that do not want to wear a smartwatch, or high-functioning patients), Android devices, sensors like Microsoft Kinect, wireless communication networks and power supplies, and data capture technology for processing and decision support or any conventional or non-conventional device/sensor performing similar functions can be and/or be included in the automated monitoring device. The automated monitoring device used herein may also comprise one or more early warning algorithm, alerting unit and a storage unit for storing data regarding one or more alerts provided by the alerting unit, i.e. previous detections increase in the sympathetic nervous activities, data about the patient, predetermined acceptable ranges and thresholds etc. In another embodiment, the automated monitoring device may also comprise of a display unit for displaying the stored data or measured values of one or more parameters. The automated monitoring device may preferably have all the units located within the same small casing to enable portability. The automated monitoring device may, for example, be embodied as a wearable device such as a bracelet, watch, anklet, shoe, armband, thigh band or a mitten.

In some embodiments, the automated sensoring device records the data measured on integrated physiological parameters such as EDA or resting EEG, in an internal memory, and further, filtering the data signals and eliminates the noises such as spikes and non-contact values (to avoid the risk that positive emotions such as joy and happiness may result in an increase in EDA as well) and obtained a baseline value. The baseline value is calculated for a patient to statistically classify any change in the physiological parameters such as EDA and/or resting EEG levels etc. on a defined scale (from 0 to 5). The term “baseline” in medicine is information found at the beginning of a study or other initial known value which is used for comparison with later data. The concept of a baseline is essential to the daily practice of medicine in order to establish a relative rather than absolute meaning to data. PANS S-EC aka PEC for patients affected with schizophrenia, BI are used as a baseline for validation of the sensoring device measure.

An algorithm can be used to determine when the patient is likely to become agitated based on these detected physiological signals. The signal can be used to determine when a patient should receive an anti-agitation agent in order to prevent agitation from emerging. The early warning algorithm can be used with both adult (including older patients) and pediatric patients. The algorithm used herein utilizes one or more than one physiological parameter from the patient, including cardiovascular signals and locomotor activity. Cardiovascular signals including EDA data, resting ECG signal data, heart rate levels, noninvasive blood pressure measurements etc. Locomotor activity can be assessed using common measuring devices such as actigraphy. Algorithms can be created that use these biometric signals to determine if a person may soon become agitated.

In some embodiments, the term “caregiver” herein refers to a person who gives care to patients who are affected with neuropsychiatric, neurodegenerative or other nervous system related diseases and are in need of taking help in care of themselves, patients suffering from opioid, alcohol or substance abuse withdrawal (including cocaine, amphetamine), or patients undergoing an OPD/IPD procedure. Caregivers can be, for example, health professionals, family members, friends, or social workers, and depending on the subject's circumstances, may give care at home or in a hospital or other healthcare setting.

An implementation of the present disclosure includes an additional technology such as mobile applications having an interface to collect an observer's feedback. Dedicated sensors may be added for additional data collection. In some implementations, systems described in the present disclosure use an Ecological Momentary Assessment (EMA). The assessment can include emotions and behaviors of a subject being repeatedly collected in everyday basis life, using of wearable electronic devices or user equipments capable of collecting data related to such as and not limited to sympathetic nervous system activity. The repeated measurements of data are for analyzing important characteristics of the dynamics of phenomena.

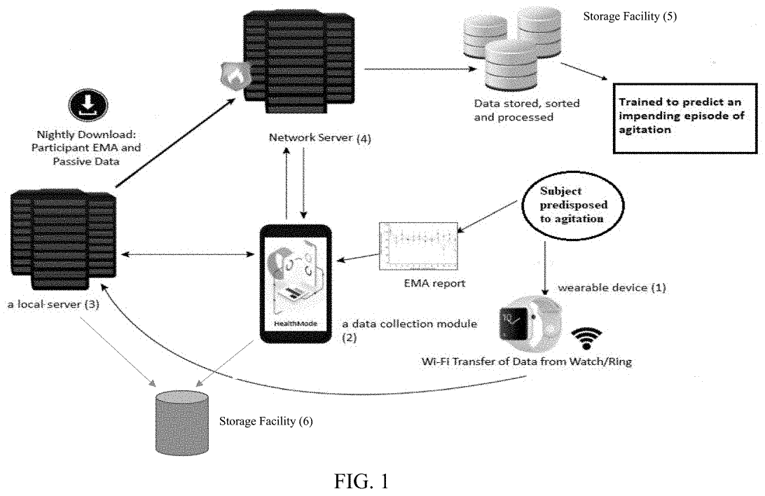

Reference is made to a system disclosed in FIG. 1 of the present disclosure. As depicted, a subject predisposed to agitation wears a wearable device for collecting data related to such as and not limited to sympathetic nervous system activity. The data collected by the wearable device are transmitted to at least a local server (e.g., via a network). In a network deployment, the local server in a non-limiting manner may comprise a server computer, a personal computer (PC), a tablet PC, a laptop computer, a desktop computer, a control system, or any machine capable of executing a set of instructions (sequential or otherwise) that specify actions to be taken by the local server. The local server includes a processor (not shown) and a memory (not shown) operatively coupled to the processor. The processor of the local server can execute functions (e.g., code stored in the memory of the local server) as described herein as being performed by the local server. A network server (also referred to as a central server) is configured to receive data from the local server. The network server includes a processor (not shown) and a memory (not shown) operatively coupled to the processor. The processor of the network server can execute functions (e.g., code stored in the memory of the network server) as described herein as being performed by the network server. In some implementations, a single server can be used instead of both the local server and the network sever. In such implementations, the single server can combine the functions of the local server and the network server.

Communication between the devices shown and described with respect to FIG. 1 can be via a communication network. The network can be a digital telecommunication network of servers and/or compute devices. The servers and/or compute devices on the network can be connected via one or more wired or wireless communication networks (not shown) to share resources such as, for example, data storage and/or computing power. The wired or wireless communication networks between servers and/or compute devices of the network 150 can include one or more communication channels, for example, a WiFi® communication channel, a Bluetooth® communication channel, a cellular communication channel, a radio frequency (RF) communication channel(s), an extremely low frequency (ELF) communication channel(s), an ultra-low frequency (ULF) communication channel(s), a low frequency (LF) communication channel(s), a medium frequency (MF) communication channel(s), an ultra-high frequency (UHF) communication channel(s), an extremely high frequency (EHF) communication channel(s), a fiber optic commination channel(s), an electronic communication channel(s), a satellite communication channel(s), and/or the like. The network can be, for example, the Internet, an intranet, a local area network (LAN), a wide area network (WAN), a metropolitan area network (MAN), a worldwide interoperability for microwave access network (WiMAX®), a virtual network, any other suitable communication system and/or a combination of such networks.

The disclosed system includes a data collection module configured to passively collect longitudinal data from the subject who has episodes of agitation in the context of diagnosis of diseases including, for example, various neuropsychiatric and neurodegenerative diseases such as Alzheimer's disease, delirium or dementia. The data collection module includes sub-modules configured to passively collect motion, position, physiological, and audio data. The data collection module can be a processor in an automatic monitoring device (e.g., a wearable device, a smart phone, or the first monitoring device 8001 shown in FIG. 8.) The data thus collected are used to develop models of agitation. The data collection module is configured to communicate with the network server and the local server for transmission of the collected data. With the collected data, an Ecological Momentary Assessment (EMA) is conducted and a report is generated by a processing unit of the system (e.g., a processor in the network server, or a processor 802 shown in FIG. 8.) For EMA data is collected from the subject. EMA also includes providing prompts to the subject, patches and updates as well. The obtained and stored data at the network server is used for training purpose to effectively monitor and predict an episode of impending agitation. The processing unit (e.g., a processor in the network server, or a processor 802 shown in FIG. 8) is configured to diagnose an impending agitation episode in a subject and to send a signal to a compatible device monitored by, for example, a caregiver alerting the caregiver about an impending agitation episode in the subject. The signal can also be sent to a remote compatible device (not shown in FIG. 1) monitored by a caregiver alerting the caregiver to an impending agitation episode in the subject. The compatible device monitored by, for example, a caregiver is also referred to as the second monitoring device 8002 in FIG. 8.

The automated sensoring device (i.e., the wearable device (1)) includes a set of sensors, a processor, and a memory. The wearable device includes one or more units for detecting the motion and location information of the subject. For example, the unit for tracking location can be any suitable satellite-based radio navigation system, such as, for example, a satellite-based radio navigation system data (e.g., GPS) module (to track longitude and latitude), a Navigation with Indian Constellation (NavIC) module, a GLObal NAvigation Satellite System (GLONASS) module, a BeiDou module, a Galileo module, a Quasi-Zenieth module, and/or the like. For example, the motion pattern can be tracked by devices such as and not limited to an accelerometer, a compass, a Gyroscope, a pedometer. The speech of the subject can be monitored by an audio monitoring unit (e.g., as recorded by a microphone) keeping track of the audio of the subject tracked in terms of time, date or duration tracking and further includes speech pace sentiment and impulsive movements. In some implementations, the wearable device can include other units for measuring the physiological data like Heart rate (HR), Heart rate variability (HRV), respiratory rate, ECG level resting heart rate (RHR), body temperature deviation, +/−EDA, ECG and the like. The body vitals and other parameters tracking are dependent on the patient. For instance, restlessness may be a trigger for agitation in some patients while it might not be so for other patients.

In some implementations, data is not continuously monitored or analyzed during the course of the training the system. The devices and data collection module will not be used to monitor the health status of the subject. The subject will be instructed to contact their physician for any changes in their health that they experience during the study.

In some implementations, the data collection module records data continuously, periodically, and/or sporadically until battery of the device perishes. The data collection module records/collects data from the moment the wearable device (or the data collection module) is switched on and is functional in the system. In some implementations, the data collection module records while charging as well. After the wearable device (or the data collection module) restarts (by a user say for reasons such as a low battery), the data collection module triggers data collection automatically. The data upload protocol as per present disclosure includes uploading the collected data for periodic saving of data [for example, at an interval of 30 minutes]. This is done within a defined interval of time. The system may include additional memory storage facility (e.g., the storage facility (5) in FIG. 1 or additional storage facility (6), each including at least one memory to store data) to keep data on the data collection module backed up, until a batch is sent successfully. The backup data may be deleted later but, in some implementations, is deleted after successful upload. A wireless communication mode such as Wi-Fi or cellular (from the wearable device (1) and/or the data collection module (2)) is used for upload channel. Devices/interfaces in the system are authorized by means of unique credentials such as an ID for the patient. In some implementations, because there can be a continuous monitoring and transfer of data, a charging protocol for devices in the system is also defined. In some implementations, the device can be charged over-night.

The alerts are signaled when there is an impending or probable agitation episode of the patient. In some implementations, alerts are sent to the clinical supervisor and also to the caregiver (or a second monitoring device 8002 accessible by the clinical supervisor or the caregiver) but no alerts are visible for patient. In some implementations, alerts can be sent to the clinical supervisor, the caregiver, and/or the patient. Alerts can also be provided to the clinical supervisor in the event of a system failure. The said system failure includes and are not limited to data upload failed/device off; data uploaded executed via cellular; a low battery, a device permission not granted; a device is static for more than 20 hours, irregularity in data upload pattern. In some instances, the alerts can be a window flashing on a monitor of the second monitoring device 8002, a text message, a call, a sound received at the second monitoring device 8002 and/or the like.

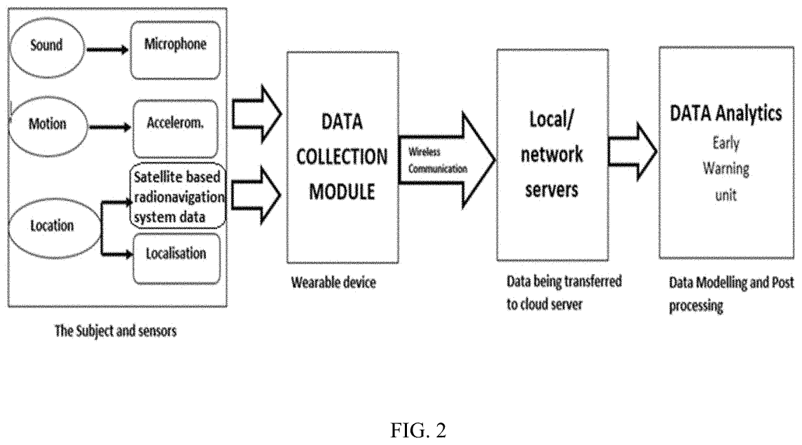

The early warning algorithm is based on machine learning. In an implementation of the disclosure is included an early warning module (included in the network server (4), or included in the memory 801 of the apparatus 800 and executable by the processor 802 in FIG. 8) implementing the said algorithm. In some implementations, the early warning module can also be included in the wearable device or the data collection module. In other words, the training of the machine learning model and the predicting/analyzing using the machine learning model can be performed by the network server, the local server, the wearable device, and/or the data collection module. The early warning module is configured to perform Data Extract, Transform and Load (ETL) Processes. Reference is made to FIG. 2 depicting an ETL process overview for an embodiment. Data is extracted from the plurality of sensors of the wearable device (1) and/or the data collection module (2). The system includes a reporting module (included in the network server (4), or included in the memory 801 of the apparatus 800 and executable by the processor 802 in FIG. 8) configured to track any issues with usage, data collection and transfer. Data processing steps occurs at various stages of the ETL process. Data processing steps may include but not limited to file compression, encryption, time stamping, and elimination of silence, speech masking or preliminary speech analysis. The data processing steps will further include data analytics providing the signals/alerts for an impending agitation of the patient.

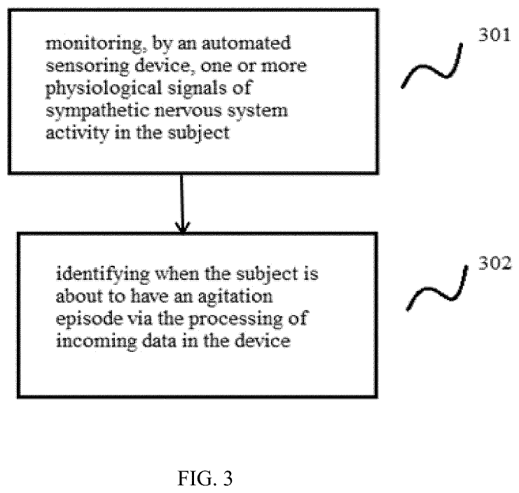

Disclosed herein is a method of diagnosing an impending agitation episode in a subject predisposed to agitation as disclosed in FIG. 3. The method comprises the following steps:

step 301: monitoring one or more physiological signals of sympathetic nervous system activity in the subject using the automated sensoring device. The automated sensoring device is placed or mounted on the subject's skin surface.

step 302: identifying when the subject is about to have an agitation episode. This is done via the processing of incoming data from the automated sensoring device. This step can be performed at the network server, the local server, or the automated sensoring device. FIG. 3 discloses an overview of the said method.

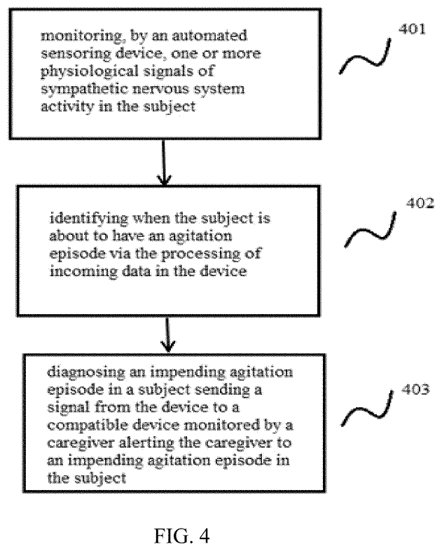

Further disclosed herein is a method of alerting a caregiver to an impending agitation episode in a subject predisposed to agitation as disclosed in FIG. 4. The said method comprises the following steps:

step 401: monitoring one or more physiological signals of sympathetic nervous system activity in the subject using an automated sensoring device placed or mounted on the subject's skin surface,

step 402: identifying, via the processing of incoming data in the automated sensoring device, when the subject is about to have an agitation episode,

step 403: diagnosing an impending agitation episode in a subject sending a signal from the automated sensoring device to a compatible device monitored by a caregiver alerting the caregiver to an impending agitation episode in the subject.

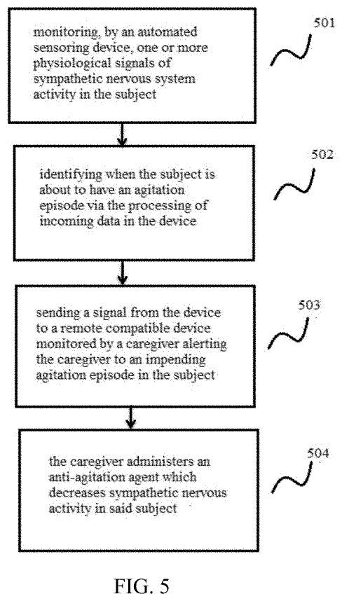

FIG. 5 shows a method of preventing the emergence of agitation in a subject predisposed to agitation. The said method comprises the following steps:

step 501: monitoring one or more physiological signals of sympathetic nervous system activity in the subject using an automated sensoring device placed or mounted on the subject's skin surface;

step 502: identifying, via the processing of incoming data in the automated sensoring device, when the subject is about to have an agitation episode;

step 503: sending a signal from the automated sensoring device to a remote compatible device monitored by a caregiver alerting the caregiver to an impending agitation episode in the subject;

step 504: administering by the caregiver an anti-agitation agent which decreases sympathetic nervous activity in said subject.

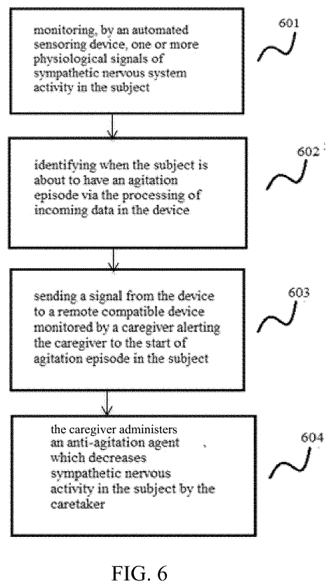

In FIG. 6 is shown a method of treating the early stage emergence of agitation or the signs of agitation in a subject predisposed to agitation. As already depicted in FIG. 6, the method comprises:

step 601: monitoring one or more physiological signals of sympathetic nervous system activity in the subject using an automated sensoring device placed or mounted on the subject's skin surface;

step 602: identifying, via the processing of incoming data in the automated sensoring device, when the subject is having an agitation episode;

step 603: sending a signal from the automated sensoring device to a remote compatible device monitored by a caregiver alerting the caregiver to the start of agitation episode in the subject and step 604: the caregiver administers an anti-agitation agent which decreases sympathetic nervous activity in said subject.

In an embodiment of the disclosure is disclosed a method of diagnosing an impending agitation episode in a subject predisposed to agitation and alerting a caregiver about the same. As already depicted in FIG. 7, the method comprises the following steps:

step 701: receiving first physiological data of sympathetic nervous system activity;

step 702: establishing a baseline value of at least one physiological parameter by training at least one machine learning model) using the first physiological data;

step 703: receiving, from a first monitoring device attached to a subject, second physiological data of sympathetic nervous system activity in the subject;

step 704: analyzing, using the at least one mathematical model (e.g., machine learning model) and based on the baseline value of at least one physiological parameter, the second physiological data to predict an agitation episode of the subject; and

step 705: sending, based on predicting the agitation episode of the subject, a signal to a second monitoring device to notify the second monitoring device of the prediction of the agitation episode of the subject such that treatment can be provided to the subject to decrease sympathetic nervous system activity in the subject.

The first monitoring device is the wearable device (e.g., smartwatch) in contact with the subject and the second monitoring device is monitored by a caregiver of the subject. The analyzing to predict the agitation episode includes determining a time period within which the agitation episode of the subject will occur and also includes determining a degree of the agitation episode of the subject.

In some embodiments, the analyzing to predict the agitation episode includes comparing the second physiological data with the baseline value of at least one physiological parameter. When the second physiological data exceeds a first threshold of the baseline value, the signal is a first signal, the treatments are first treatments while when the second physiological data exceeds a second threshold of the baseline value, the signal is a second signal different from the first signal, the treatments are second treatments different from the first treatments. For example, the machine learning model (or other mathematical model) can determine, based on the training data (i.e., the first physiological data described in FIG. 7), that when the average EEG of the subject is below a first threshold, the probability of the subject being in a calm state is high (e.g., above 80%). Moreover, for example, the machine learning model (or other mathematical model) can determine, based on the training data, that when the average EEG of the subject is between the first threshold and a second threshold, the subject is more likely to have an agitation episode in the next hour (or a pre-determined time period). The machine learning model (or other mathematical model) determines, based on the training data, that when the average EEG exceeds the second threshold, the subject is more likely having the agitation episode. Upon receiving the new EEG data of the subject, the processor (e.g., processor 802 in FIG. 8) can compare the new EEG data with the first threshold and the second threshold. When the new EEG data is between the first threshold and the second threshold, the processor predicts that the subject is more likely to have an agitation episode in the next hour. The processor can send a first signal to the second monitoring device (e.g., 8002 in FIG. 8) to alert the caregiver. Thus, first treatments can be administered to the subject on a timely basis to avoid the agitation episode. When the new EEG data exceeds the second threshold, the processor can send a second signal to the second monitoring device such that different treatments can be administered to the subject. In some instances, the thresholds can be determined by the machine learning model (or other mathematical model). In some instances, a machine learning model (e.g., a deep learning model) is used to establish the baseline value, identify anomalies and/or predict the agitation episode.

While described herein as using a trained machine learning model to analyze and predict an agitation episode, in some implementations, any other suitable mathematical model and/or algorithm can be used. For example, once a baseline is established, a mathematical model can compare subsequent patient data to the baseline to determine whether the patient data varies from the baseline by a predetermined amount and/or statistical threshold. In such an example, if the patient data varies from the baseline by the predetermined amount and/or statistical threshold, an alert can be generated and provided.

In some implementations, the second physiological data is received during a first time period. A third physiological data of sympathetic nervous system activity in the subject is received a second time period after the first time period. A report of sympathetic nervous system activity in the subject to identify a pattern of a change of sympathetic nervous system activity in the subject is generated. The report is based on the second physiological data and the third physiological data. For example, the report of sympathetic nervous system activity can show that the subject is more (or less) likely to have an agitation episode during a specific time period of a day (e.g., in the morning, after a meal), or after a specific event takes place (e.g., after a visit by a family member). Such a report of a pattern of a change (or a trend) of sympathetic nervous system activity in the subject can help the caregiver reduce the likelihood of the occurrence of the agitation episode of the subject or better prepare for the occurrence.

In some implementations, the said second physiological data of sympathetic nervous system activity can include at least one of a change in electrodermal activity, heart rate variability, cognitive assessments such as pupil size, secretion of salivary amylase, blood pressure, pulse, respiratory rate, or level of oxygen in blood. It should be noted that these have been mentioned by way of example and not by means of limitation. The factors to be monitored are also dependent on the patient. The sympathetic nervous system activity is assessed by measuring any change in electrodermal activity or any change in electrodermal activity together with any change in resting electroencephalography

The method of this embodiment further includes receiving an indication associated with the agitation episode after sending the signal to the second monitoring device and training the at least one machine learning model based on the indication.

In an embodiment of the disclosure is disclosed an apparatus (800), comprising a memory (801) and a processor (802) operatively coupled to the memory. A block diagram of the apparatus is shown in FIG. 8. In some implementations, the apparatus (800) is similar structurally and functionally to the network server (4) and/or the local server (3) in FIG. 1. The said processor is configured to receive, from a first monitoring device (8001) attached to a subject, physiological data of sympathetic nervous system activity in the subject. The first monitoring device (8001) is an automated monitoring device. The processor is capable of analyzing the physiological data to detect an anomaly from a reference pattern of sympathetic nervous system activity to determine a probability of an occurrence of an agitation episode of the subject. For the purpose, the processor executes at least one machine learning model. The processor (802) is further capable of sending a signal to a second monitoring device (8002) to notify the second monitoring device of the probability of the occurrence of the agitation episode of the subject such that treatment can be provided to decrease sympathetic nervous system activity in the subject. The second monitoring device is a device monitored by the caregiver (e.g., remote from the subject). The second monitoring device may be an end user terminal capable of alerting the caregiver by means of the sound/alarm and/or display. The second monitoring device may be and not limited to a computer or a smart phone.

The processor (802) is configured to receive an indication associated with the agitation episode after sending the signal to the second monitoring device and further train the at least one machine learning model based on the indication. The said indication indicates one of (1) whether or not the agitation episode occurs, (2) when the agitation episode occurs, (3) a degree of the agitation episode, (4) a time period for which the agitation episode lasts, or (5) a symptom of the agitation episode.

The machine learning models (or other mathematical models) can be trained using supervised learning and unsupervised learning. The machine learning model (or other mathematical models) of the apparatus (800) is trained based on at least one of supervised learning, unsupervised learning, semi-supervised learning, and/or reinforcement learning. In some implementations the supervised learning can include a regression model (e.g., linear regression), in which a target value is found based on independent predictors. This follows that the said model is used to find the relation between a dependent variable and an independent variable. The at least one machine learning model may be any suitable type of machine learning model, including, but not limited to, at least one of a linear regression model, a logistic regression model, a decision tree model, a random forest model, a neural network, a deep neural network, and/or a gradient boosting model. To predict an agitation episode, the processor is configured to analyze the data. For the purpose, the processor is configured to determine, based on a comparison between the second physiological data and the baseline value, a degree of the agitation episode of the subject. The machine learning model (or other mathematical model) can be software stored in the memory 801 and executed by the processor 802 and/or hardware-based device such as, for example, an ASIC, an FPGA, a CPLD, a PLA, a PLC and/or the like. In some implementations, the apparatus (800) is similar structurally and functionally to the network server (4) and/or the local server (3) in FIG. 1.

In some implementations a non-transitory machine-readable medium storing code representing instructions to be executed by a processor can be used. The instructions may further be transmitted or received over a network via the network interface device. The term “machine-readable medium” shall be taken to include any medium that is capable of storing, encoding or carrying a set of instructions for execution by the machine and that cause the machine to perform any one or more of the methodologies of the present disclosure. The term “machine-readable medium” shall accordingly be taken to include, but not be limited to: tangible media; solid-state memories such as a memory card or other package that houses one or more read-only (non-volatile) memories, random access memories, or other re-writable (volatile) memories; magneto-optical or optical medium such as a disk or tape; non-transitory mediums or other self-contained information archive or set of archives is considered a distribution medium equivalent to a tangible storage medium. Accordingly, the disclosure is considered to include any one or more of a machine-readable medium or a distributed medium, as listed herein and including art-recognized equivalents and successor media, in which the software implementations herein are stored. The said code comprises code to cause the processor to perform the function. The said code comprises code to cause the processor to train, prior to analyzing using the at least one mathematical model (e.g., machine learning model), the at least one mathematical model (e.g., machine learning model) based on training physiological data of sympathetic nervous system activity associated with a plurality of subjects. The at least one mathematical model (e.g., machine learning model) includes a plurality of physiological parameters as input. Each physiological parameter from the plurality of physiological parameters is associated with a weight from a plurality of weights of the mathematical model (e.g., machine learning model). The medium includes code to cause the processor to determine the reference pattern of at least one physiological parameter from the plurality of physiological parameters based on the at least one mathematical model (e.g., machine learning model). The code includes code to cause the processor to receive an indication associated with the agitation episode after sending the signal to the second monitoring device and thus train the at least one mathematical model (e.g., machine learning model) to adjust the reference pattern of the at least one physiological parameter and a weight associated with the at least one physiological parameter.

In some implementations, the memory 801 can store a mathematical model database and/or a machine learning model database (not shown), which may include the physiological data of sympathetic nervous system activity of the subject, any additional data (e.g., location, motion, audio, accelerometer, gyroscope, compass, satellite-based radio navigation system data, and/or any data received from the first monitoring device 8001 (or sensors from the first monitoring device 8001) and/or patient data. The patient data can include patient medical data (e.g., demographics, medical history, type of cancer, stage of cancer, previous treatments and responses, progression history, metabolomics, and/or a histology). In some implementations, the physiological data of sympathetic nervous system activity, additional data of sympathetic nervous system activity, and/or the patient data can be used to train a machine learning model (or other mathematical model).

In some implementations, the processor 802 can receive first physiological data of sympathetic nervous system activity during a first time period. The processor 802 can establish a reference pattern (including at least one baseline value or threshold) by training the machine learning model (or other mathematical model) based on the first physiological data. During a second time period after the first time period, the processor 802 can receive second physiological data and analyze the second physiological data using the machine learning model (or other mathematical model) to identify the anomaly and/or predict the agitation episode. The training step (e.g., step 702 in FIG. 7) and the analyzing step (e.g., step 704 in FIG. 7) can be performed by the processor 802 or different processors. In some instances, the first physiological data and the second physiological data can be associated with a single subject (e.g., collected by monitoring the subject during a monitoring phase and/or time period). In some instances, the first physiological data can be associated with a set of subjects including or not including the subject from which the second physiological data are received. In some instances, the first physiological data are training data used by the machine learning model (or other mathematical model) to establish the reference pattern. The training data can be the data specific or personalized to the subject and based on monitoring the subject for a training period. In some instances, the training data can be associated with other similar subjects (e.g., with similar characteristics, demographics, medical history, etc.). In some instances, the training data can be based on feedback or indications when (or after) the agitation episodes occur.

In some implementations, the processor 802 can receive an indication after sending the signal to alert the prediction of the agitation episode. For example, the caregiver can provide the indication to the processor 802 of whether or not the predicted agitation episode has happened, the intensity level of the agitation episode, the time at which the agitation episode happens, the duration of the agitation episode, and/or other characteristics of the agitation episode. Based on the indication received, the processor 802 can further train the machine learning model (or other mathematical model) through reinforcement learning. Specifically, the processor 802 can fine tune the set of physiological parameters and/or the weight(s) associated with the machine learning model (or other mathematical model) so that the machine learning model (or other mathematical model) can provide more accurate predictions.

In some implementations, the processor 802 can be, for example, a hardware based integrated circuit (IC) or any other suitable processing device configured to run and/or execute a set of instructions or code. The processor 802 can be configured to execute the process described with regards to FIG. 7. For example, the processor 802 can be a general purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 802 is operatively coupled to the memory 801 through a system bus (for example, address bus, data bus and/or control bus).

The memory 801 can be, for example, a random access memory (RAM), a memory buffer, a hard drive, a read-only memory (ROM), an erasable programmable read-only memory (EPROM), and/or the like. The memory 801 can store, for example, one or more software modules and/or code that can include instructions to cause the processor 801 to perform one or more processes, functions, and/or the like (e.g., the machine learning model). In some implementations, the memory 801 can be a portable memory (for example, a flash drive, a portable hard disk, and/or the like) that can be operatively coupled to the processor 802.

### Therapeutic Agents:

Any anti-agitation agent that can reduce sympathetic nervous system activity may be used as part of the system herein to prevent the emergence of agitation. One particular group of suitable agents are alpha-2-adrenergic receptor agonists.

Alpha-2 adrenergic receptor agonists:

Microbiologists have been able to subdivide the various classes of α-2 receptors based upon affinities for agonists and antagonists. The α-2 receptors constitute a family of G-protein—coupled receptors with three pharmacological subtypes, α-2A, α-2B, and α-2C. The α-2A and -2C subtypes are found mainly in the central nervous system. Stimulation of these receptor subtypes may be responsible for sedation, analgesia, and sympatholytic effects (Joseph A. Giovannitti, Jr et al. Alpha-2 Adrenergic Receptor Agonists: A Review of Current Clinical Applications, Anesthesia Progress, 2015).

In one embodiment, the alpha-2 adrenergic receptor agonist includes, but is not limited to, clonidine, guanfacine, guanabenz, guanoxabenz, guanethidine, xylazine, tizanidine, medetomidine, dexmedetomidine, methyldopa, methylnorepinephrine, fadolmidine, iodoclonidine, apraclonidine, detomidine, lofexidine, amitraz, mivazerol, azepexol, talipexol, rilmenidine, naphazoline, oxymetazoline, xylometazoline, tetrahydrozoline, tramazoline, talipexole, romifidine, propylhexedrine, norfenefrine, octopamine, moxonidine, lidamidine, tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof.

In a preferred embodiment, the alpha-2 adrenergic receptor agonist is dexmedetomidine or a pharmaceutically acceptable salt thereof, especially the hydrochloride salt.

Dexmedetomidine hydrochloride, also known in the intravenous form as Precedex®, is a highly selective α2-adrenergic agonist. It is the pharmacologically active d-isomer of medetomidine (Joseph A. Giovannitti, Jr et al. Alpha-2 Adrenergic Receptor Agonists: A Review of Current Clinical Applications, Anesthesia Progress, 2015). Unlike other sedatives such as benzodiazepines and opioids, dexmedetomidine achieves its effects without causing respiratory depression. Dexmedetomidine exerts its hypnotic action through activation of central pre- and postsynaptic α2-receptors in the locus coeruleus. PRECEDEX® has been approved by the US FDA for use in ICU sedation, namely sedation of initially intubated and mechanically ventilated patients during treatment in an intensive care settings, and procedural sedation, namely sedation of non-intubated patients prior to and/or during surgical and other procedures, and is known to be a safe and effective sedative.

In WO 2018/126182, the disclosure of which is incorporated herein by reference, we describe the treatment of agitation or the signs of agitation in a subject by sublingually administering dexmedetomidine or a pharmaceutically acceptable salt thereof. Advantageously, agitation is effectively treated without also causing significant sedation. In a preferred embodiment, the present disclosure provides a sublingual dexmedetomidine hydrochloride product, such as a thin film, to reduce sympathetic nervous system activity as part of the system herein to prevent the emergence of agitation. In a particular embodiment, the system prevents the emergence of agitation without also causing significant sedation.

Agitation in patients with neuro-psychiatric or neuro-degenerative diseases results in patients that are uncooperative to treatment, and are also potentially violent and aggressive, making them a danger to themselves and to caregivers. By detecting a signal that indicates a patient is about to become agitated, the present disclosure pairs a diagnostic with a treatment component using an anti-agitation drug, such as an alpha2 adrenergic agonist like dexmedetomidine, to prevent the manifestation of an agitation episode. Thus, according to the present disclosure, dexmedetomidine can be used as a prophylactic or preventive therapeutic agent.

Monitoring Devices/Sensors:

A wide range of devices/sensors, such as suitable sensor device such as, for example, a waist worn multi-sensor device with networking capability, a wrist worn multi-sensor device with networking capability, a finger worn multi-sensor device with networking capability, and/or the like. In specific embodiments, wide range of devices/sensors, such as, for example, a smartphone (e.g., iPhone (BYOD or provisioned), accelerometers and gyroscopes, portable devices, digital devices, smart fabrics, bands and actuators like an smart watch [e.g., Apple watch (e.g., Apple watch 3) or iWatch], smart patch such as MC10 Patch, Oura rings particularly for patients unable or that do not want to wear a smartwatch, or high-functioning patients, Android devices, sensors like Microsoft Kinect, wireless communication networks and power supplies, and data capture technology for processing and decision support or any conventional or non-conventional device/sensor performing similar functions can fall under this defined term. Oura Cloud API is a collection of HTTP REST API endpoints and uses OAuth2 for authentication. The device used herein may also comprise one or more early warning algorithm, alerting unit and a storage unit for storing data regarding one or more alerts provided by the alerting unit, i.e. previous detections increase in the sympathetic nervous activities, data about the patient, predetermined acceptable ranges and thresholds etc.

In some embodiments, the automated sensoring device records the data measured on integrated parameters including physiological parameters such as EDA or resting EEG, motion parameters and audio parameters in an internal memory, and further, filters the data signals and eliminates noise such as spikes and non-contact values (to avoid the risk that positive emotions such as joy and happiness may result in an increase in EDA as well). The baseline value can be calculated for a patient to statistically classify any change in the physiological parameters such as EDA and/or resting EEG levels etc. on a defined scale (from 0 to 5).

Methods:

The present disclosure provides a method of detecting the signs of emergence of agitation in a subject using a monitoring device that measures the change in the physiological signals that arise due to increased sympathetic nervous activity in the subject, indicative of an impending agitation episode.

The present disclosure also provides a method of alerting a caregiver to the signs of emergence of agitation in a subject via an interface between the device that measures the change in the physiological signals that arise due to the increased sympathetic nervous activity and a suitable compatible device, such as an end-user display terminal. The method involves the device signaling information related to increases in sympathetic nervous system activity, e.g. remotely via Bluetooth, to a receiving unit, such as an end-user display terminal, which may then actively alert the caregiver to an impending agitation episode or may passively present (e.g. display on a screen) the information received from the device for review and action by the caregiver.

The present disclosure also provides a method of preventing the emergence of agitation in a subject, wherein the caregiver assesses the information received from the aforementioned device and takes action to calm the subject, such as by administering to the subject an anti-agitation agent that decreases the sympathetic nervous system activity in the subject.

In some embodiments, the device monitors the change in sympathetic nervous system activity by measuring EDA over time. The device may also monitor other physiological signals, including heart rate variability such as resting EEG, cognitive assessments such as pupil size, secretion of salivary amylase, blood pressure; pulse; respiratory rate, level of oxygen in the blood and other signals related to increased sympathetic nervous system activity.

In some embodiments, the automated sensoring device records and collect objective data on integrated physiological parameters (such as EDA, resting EEG, blood pressure, mobility/motor, memory/processing, speech/sleep patterns, social engagement, etc.) in an internal memory of the device and utilize algorithms to transform the data into a format that is interpretable as a specific measure, or, an aggregate functional outcome, including, filtering the data signals and eliminates the noises such as spikes and non-contact values (to avoid the risk that positive emotions such as joy and happiness may result in an increase in EDA as well) and obtains a baseline value. The baseline value is calculated for a patient to statistically classify any change in the physiological parameters such as EDA and/or resting EEG levels etc. on a defined scale (from 0 to 5). PANSS-EC aka PEC for patients affected with schizophrenia, Bipolar disorder are used as a baseline for validation of the sensoring device measure. The present disclosure utilizes predictive algorithms and provides related wearable device technology that enable the administration of dexmedetomidine or a pharmaceutically acceptable salts prior to the onset of an agitation episode, which, may reduce the burden on the patient and caregiver. In preferred embodiment, dexmedetomidine is in the form of thin sublingual film. Suitable thin sublingual films containing dexmedetomidine are described in PCT Application No. PCT/US2019/039268 and incorporated here by reference. In some embodiments the automated monitoring device sends/transfer signals to a computer database through a Bluetooth or any other transmission-related technology.

In a particular embodiment, signs of emergence of agitation are detected by monitoring EDA with the help of the automated sensoring device placed on the skin of the patient. The said device monitors the EDA by recording the changes in the patient's skin's electrical resistance, since any change in sympathetic nervous system activity results in a slight increase in perspiration, which lowers skin resistance (because perspiration contains water and electrolytes) and sends the data in an internal memory of the device and further transfer the collected data to a computer database that includes a plurality of early warning algorithms and transform the data into a format that is interpretable as a specific measure, or, an aggregate functional outcome, including, filtering the data signals and elimination the noises such as spikes and non-contact values (to avoid the risk that positive emotions such as joy and happiness may result in an increase in EDA as well) and obtained a baseline value.

In some embodiment, the patient monitoring device includes at least one patient monitor that includes a display device and at least one sensor connected to the patient to obtain physiological data from the patient. The patient monitoring device is further connected to a computer database that includes one or more of early warning algorithms. Each of the early warning algorithms operates to predict the early signs the emergence of agitation of a patient based upon multiple parameters of physiological data and then generates patient alerts/warnings based upon the operation of the early warning algorithm.

In some embodiments, the process of generating early warning algorithm includes 3 stages namely development stage 1; development stage 2; development stage 3.

Development stage 1 can include the steps of creation of (i) data collection tools (ii) data processing tools (iii) infrastructure. Data collection tool includes validation of passive and active mobile data collection tools in terms of usability, user experience, patient engagement and needs; determination of reliability of used hardware sensors for continuous motion (e.g. accelerometer, gyroscope, compass, pedometer, activity type, physical performance, location, satellite-based radio navigation, etc.), physiological and audio data collection (e.g. recognition of speech pace sentiment and impulsive movements). And make necessary improvements to engaged data collection tools. Data processing tools includes building of basic classification model prototypes for: i) motion processing ii) audio processing iii) physiological state processing, based on reference data and observation of achieved performance of models and document edge cases. Infrastructure includes defining and implementing a scalable, plug-and-play system architecture for real-time mobile-based data collection, processing, interpretation and communication, as building an early warning system for acute patient state demands it.

Development stage 2 includes steps of research integration and classification model improvement. Research integration include data collation, expert annotation, data curation and model training. Classification model improvement including improving performance in specificity and sensitivity of descriptive models per use case: i) motion, audio, physiological data, ii) in vs. out-hospital, iii) broadening TA applicability. Model improvement further includes developing first symptom-occurrence prediction models and developing first patient-specific agitation profiles based on: i) type, length and intensity of 3 stages: onset, episode and recovery, (ii) episode frequency and concurrence.

Development stage 3 includes steps of research integration and classification model improvement. Research integration includes comparing an acute agitation measure with established assessment methods (PANS S-EC). Classification model improvement include improving performance of predictive models in specificity and sensitivity per use case: i) motion, audio, physiological data, ii) in vs. out-hospital, iii) broadening therapeutic area applicability (continuous cycles). It also includes augmenting the engine creating patient-specific agitation profiles by predictive features (aimed at progression and prognosis

In some embodiments, signs of emergence of agitation are monitored in patients suffering from neuropsychiatric diseases selected from the group comprising of schizophrenia, bipolar disorder, bipolar mania, delirium, major depressive disorder, depression and other related neuropsychiatric diseases. In some instances, patient is suffering from schizophrenia or delirium, preferably schizophrenia. In some embodiments, signs of emergence of agitation are monitored in patients suffering from delirium. The various instruments used for measuring agitation in delirium patients include Richmond Agitation and Sedation Scale (RASS), Observational Scale of Level of Arousal (OSLA), Confusion Assessment Method (CAM), Delirium Observation Screening Scale (DOS), Nursing Delirium Screening Scale (Nu-DESC), Recognizing Acute Delirium As part of your Routine (RADAR), 4AT (4 A's Test). In some embodiments, signs of emergence of agitation are monitored in patients suffering from bipolar disorder. The various instruments used for measuring agitation in bipolar disorder patients include Positive and Negative Syndrome Scale-Excited Component (PANS S-EC), Montgomery-Åsberg Depression Rating Scale (MADRS), single-item Behavioral Activity Rating Scale (BARS). In some embodiments, signs of emergence of agitation are monitored in patients suffering from neurodegenerative disease, such as Alzheimer's disease, frontotemporal dementia (FTD), dementia, dementia with Lewy bodies (DLB), post-traumatic stress disorder, Parkinson's disease, vascular dementia, vascular cognitive impairment, Huntington's disease, multiple sclerosis, Creutzfeldt-Jakob disease, multiple system atrophy, traumatic brain injury or progressive supranuclear palsy. In some embodiments, signs of emergence of agitation are monitored in patients suffering from dementia. The various instruments used for measuring agitation in dementia patients include Cohen-Mansfield Agitation Inventory (CMAI), Agitated behavior scale (ABS), battery of scales for dementia (e.g.; BAS, ABID, MPI) could be used as a baseline for validation of the new digital measure such as Middelheim Frontality Score (MFS), Behavioral Pathology in Alzheimer's Disease Rating Scale (Behave-AD), Cornell Scale for Depression in Dementia (CSDD).

In some embodiments, signs of emergence of agitation are monitored in patients suffering from opioid, alcohol and substance abuse withdrawal (including cocaine, amphetamine).

In some embodiments, signs of emergence of agitation are monitored in patients undergoing OPD/IPD procedures (e.g. MM, CT or CAT scan, lumbar puncture, bone marrow aspiration biopsy, tooth extraction or other dental procedures).

In some embodiments, the present disclosure provides a method of preventing the emergence of agitation in a subject predisposed to agitation comprising:
(a) monitoring one or more physiological signals of sympathetic nervous system activity in the subject using an automated sensoring device placed or mounted on the subject's skin surface;
(b) identifying, via the processing of incoming data in the device, when the subject is about to have an agitation episode;
(c) sending a signal from the device to a remote compatible device monitored by a caregiver alerting the caregiver to an impending agitation episode in the subject; and
(d) administering by the caregiver dexmedetomidine or a pharmaceutically acceptable salt thereof to reduce sympathetic nervous activity in said subject.

In a particular embodiment, dexmedetomidine or a pharmaceutically acceptable salt thereof, for example dexmedetomidine hydrochloride, is administered sublingually, for example via a thin film, to the subject. In some instances, the emergence of agitation is prevented without also causing significant sedation.

In some embodiments, increase in sympathetic nervous activity is detected by measuring the electrodermal activity wherein, the monitoring device is clipped to the finger of a patient with attaching electrodes to the middle phalanges of adjacent fingers of a hand and measuring/analyzing EDA waveforms. The data obtained by the clipped device is then transferred to the computer database, connected the monitoring device, wherein the computer database includes one or more of early warning algorithms. Based on the data analyzed, early warning algorithms operates to predict the early signs the emergence of agitation of a patient and generates patient alerts/warnings based upon the operation of the early warning algorithm to the caregiver that an anti-agitation agent should be administered.

In a particular embodiment, conveniently, a clipped device can be a commercial device, such as a Biopac MP150 system, is used to monitor EDA. Here, 11-mm inner diameter silver/silver chloride electrodes filled with isotonic electrode paste are attached to the middle phalanges of the fourth and fifth fingers of the non-dominant hand. EDA waveforms are analyzed with AcqKnowledge software or Matlab, with base-to-peak differences assessed for the largest deflection in the window one to four seconds following stimulus onset.

In another embodiment, increase in sympathetic nervous activity is detected by measuring a resting EEG in a patient. For example, the patient wears an electrode cap containing multiple scalp electrodes, e.g. ranging from about 3 to about 128 electrodes. The cap includes 1 ground electrode placed above the forehead, and a set of linked reference electrodes, one placed on each ear lobe. Vertical and horizontal electro-oculograms (VEOG and HEOG) are recorded and used to collect EEG data for eye blink and eye movement. EEG activity (e.g. spectral power, topographic microstate, and interelectrode coherence) during wakeful rest are also monitored. Recordings of monitored data is obtained for up to three minutes of closed-eye resting EEG. Patients are told to relax with eyes closed for the session and told to remain as still as possible (to minimize movement artifacts in the EEG).

In some embodiments, the monitoring device monitors the resting EEG and then transferred the obtained data to the computer database, connected the monitoring device, wherein the computer database includes one or more of early warning algorithms. Based on the data analyzed, early warning algorithms operates to predict the early signs the emergence of agitation of a patient and generates patient alerts/warnings based upon the operation of the early warning algorithm to the caregiver that an anti-agitation agent should be administered.

In a particular embodiment, both EDA and resting EEG are monitored to determine if the subject is about to have an agitation episode.

In some embodiments, sympathetic nervous system activity is monitored by audio, motion and physiological signals. Audio signals can include, for example, tearfulness, talking more quickly than average, outbursts of shouting, incessant talking and incoherent speech. Motion signals can include, for example, dominant hand (fidgeting, taping fingers/hands, hand-wringing, nail-biting, picking at skin); body (chaotic body positioning changes, Taping feet, Shuffle), body and hand (inability to sit still, general restlessness, pacing & wondering (e.g. around a room), starting/stopping tasks abruptly, taking off clothes then put them back on). Physiological signals can include, for example, change in skin conductance (GSR); electrodermal activity (EDA), temperature variability (skin temperature), electromyography (EMG) levels, heart rate variability such as resting EEG, ECG; actigraphy/polysomnography; cognitive assessments such as pupil size; secretion of salivary amylase; blood pressure; pulse rate; respiratory rate; level of oxygen in the blood and any other signal related to sympathetic nervous system activity. There are some composite signals include some blend of motion audio physiological data) such as extreme irritability, exasperation and anger, excessive excitement, mood swings or the like.

In a further embodiment, the present disclosure provides a method of preventing the emergence of agitation in a subject with schizophrenia comprising:
(a) monitoring one or more signals (physiological, motion or audio) of sympathetic nervous system activity in the subject using an automated sensoring device placed or mounted on the subject's skin surface;
(b) identifying, via the processing of incoming data in the device, including EDA data, when the subject is about to have an agitation episode;
(c) sending a signal from the device to a remote compatible device monitored by a caregiver alerting the caregiver to an impending agitation episode in the subject; and
(d) administering by the caregiver dexmedetomidine or a pharmaceutically acceptable salt thereof to reduce sympathetic nervous activity in said subject.

In another embodiment, the present disclosure provides a method of preventing the emergence of agitation in a subject with dementia comprising:
(a) monitoring one or more signals (physiological, motion or audio) of sympathetic nervous system activity in the subject using an automated sensoring device placed or mounted on the subject's skin surface;
(b) identifying, via the processing of incoming data in the device, including EDA and resting EEG data, when the subject is about to have an agitation episode;
(c) sending a signal from the device to a remote compatible device monitored by a caregiver alerting the caregiver to an impending agitation episode in the subject; and
(d) administering by the caregiver dexmedetomidine or a pharmaceutically acceptable salt thereof to reduce sympathetic nervous activity in said subject.

In a further embodiment, the present disclosure provides a method of preventing the emergence of agitation in a subject with delirium comprising:
(a) monitoring one or more signals (physiological, motion or audio) of sympathetic nervous system activity in the subject using an automated sensoring device placed or mounted on the subject's skin surface;
(b) identifying, via the processing of incoming data in the device, including EDA data, when the subject is about to have an agitation episode;
(c) sending a signal from the device to a remote compatible device monitored by a caregiver alerting the caregiver to an impending agitation episode in the subject; and
(d) administering by the caregiver dexmedetomidine or a pharmaceutically acceptable salt thereof to reduce sympathetic nervous activity in said subject.

In one embodiment, the automated sensoring device is wearable digital device. In more some embodiments, the wearable device is wrist worn multi-sensor device with networking capability (e.g., wearable watch such as Apple watch). The present disclosure also provides a method of preventing the emergence of agitation in a subject identified by measuring one or more physiological signals of sympathetic nervous system activity as about to have an agitation episode, comprising administering to the subject an effective amount of an alpha-2 adrenergic receptor agonist or a pharmaceutically acceptable salt thereof, preferably dexmedetomidine or a pharmaceutically acceptable salt thereof Further, the present disclosure provides prevention and treatment of agitation comprising the administration of dexmedetomidine or a pharmaceutically acceptable salt therefore prior to the onset of agitation.

In another embodiment, the present disclosure provides a method of preventing the emergence of agitation in a subject identified by measuring one or more physiological signals of sympathetic nervous system activity as well as motor system activity as about to have an agitation episode, comprising administering sublingually to the subject an effective amount of an alpha-2 adrenergic receptor agonist or a pharmaceutically acceptable salt thereof, preferably dexmedetomidine or a pharmaceutically acceptable salt thereof.

In another embodiment, the present disclosure provides a method of preventing the emergence of agitation in a subject identified by measuring one or more physiological signals of sympathetic nervous system activity as well as motor system activity as about to have an agitation episode, comprising administering to said subject a sublingual film product, where the sublingual film product comprises an effective amount of an alpha-2 adrenergic receptor agonist or a pharmaceutically acceptable salt thereof, preferably dexmedetomidine or a pharmaceutically acceptable salt thereof.

In a further embodiment, the emergence of agitation is prevented without inducing concomitant significant sedation.

Pharmaceutical Compositions, their Preparation and Administration:

Anti-agitation agents, including alpha-2 adrenergic receptor agonists such as dexmedetomidine or a pharmaceutically acceptable salt thereof, may be used in the present disclosure to prevent agitation in the form of pharmaceutical compositions suitable for oral, parenteral (including subcutaneous, intradermal, intramuscular, intravenous, intraarticular, and intramedullary), transmucosal (sublingual or buccal), intraperitoneal, transdermal, intranasal, rectal and topical (including dermal) administration. In a preferred embodiment, the route of administration of an alpha-2 adrenergic receptor agonist such as dexmedetomidine or a pharmaceutically acceptable salt thereof is transmucosal, especially sublingual.

The composition may conveniently be presented in a unit dosage form and may be prepared by any of the methods well known in the art of pharmacy. Typically, these methods include the step of bringing into association the active ingredient (e.g. an alpha-2 adrenergic receptor agonist such as dexmedetomidine or a pharmaceutically acceptable salt thereof) with the carrier which constitutes one or more accessory ingredients.

The pharmaceutical composition may be formulated as an injection, tablet, capsule, film, wafer, patch, lozenge, gel, spray, liquid drops, solution, suspension and the like. In a preferred embodiment, the composition is a sublingual film, particularly when the active ingredient is an alpha-2 adrenergic receptor agonist such as dexmedetomidine or a pharmaceutically acceptable salt thereof.

Various processes can be used for manufacturing tablets according to the disclosure. Thus, for example, the active ingredient may be dissolved in a suitable solvent (with or without binder) and distributed uniformly over lactose (which may contain other materials), to prepare granules, e.g. by a known granulation, coating or spraying process. Granules can be sized via sieving and/or further processed by a dry granulation/slugging/roller compaction method, followed by a milling step to achieve suitable granules of specific particle size distribution. The sized granules may then to be blended with other components and/or and lubricated in a suitable blender and compressed into tablets of specific dimensions using appropriate tooling.

Compositions suitable for parenteral administration include aqueous and non-aqueous sterile injection solutions, which may contain anti-oxidants, buffers, bacteriostatic agent and solutes to render the formulation isotonic with the blood of the intended recipient. Aqueous and non-aqueous sterile suspensions may include, for example, suspending, thickening and/or wetting agents (such as, for example, Tween 80). The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

In one particular embodiment, the anti-agitation composition used in the present disclosure to prevent agitation is PRECEDEX®.

For application topically to the skin, the pharmaceutical composition may conveniently be formulated with a suitable ointment containing the active component suspended or dissolved in a carrier. Carriers for topical administration include, but are not limited to, mineral oil, liquid petroleum, white petroleum, propylene glycol, polyoxyethylene polyoxypropylene compound, emulsifying wax and water. Alternatively, the pharmaceutical composition may be formulated as a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. Transdermal patches and iontophoretic administration are also included in this disclosure.

The pharmaceutical compositions may also be administered in the form of suppositories for rectal administration. These compositions can be prepared by mixing the active ingredient with a suitable non-irritating excipient which is solid at room temperature but liquid at the rectal temperature and therefore will melt in the rectum to release the active component. Such materials include, but are not limited to, cocoa butter, beeswax and polyethylene glycols.

The pharmaceutical compositions may also be administered intra-nasally or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In one particular embodiment, the anti-agitation composition used in the present disclosure to prevent agitation is an intra-nasal spray, particularly a spray comprising dexmedetomidine or a pharmaceutically acceptable salt thereof, for example, as described in International patent application publication WO 2013/090278A2, the contents of which are herein incorporated by reference.

In a preferred embodiment, the pharmaceutical composition is a sublingual composition that may comprise a pharmaceutically acceptable carrier. Suitable pharmaceutically acceptable carriers include water, sodium chloride, binders, penetration enhancers, diluents, lubricants, flavouring agents, coloring agents and so on.

The sublingual composition can be, for example, a film, wafer, patch, lozenge, gel, spray, tablet, liquid drops or the like. In one embodiment, the sublingual composition is in the form of a tablet or packed powder.

In one particular embodiment, the anti-agitation composition used in the present disclosure to prevent agitation is a sublingual (or buccal) spray, particularly a spray comprising dexmedetomidine or a pharmaceutically acceptable salt thereof, for example, as described in International patent application publication WO 2010/132882A2, the contents of which are herein incorporated by reference.

In a preferred embodiment, the sublingual composition is a film (e.g. a thin film), particularly a film comprising dexmedetomidine or a pharmaceutically acceptable salt thereof. In a particular embodiment, the film is a self-supporting, dissolvable, film, comprising: (i) dexmedetomidine or a pharmaceutically acceptable salt thereof; (ii) one or more water-soluble polymers; and, optionally, (iii) one or more pharmaceutically acceptable carriers. In a preferred aspect, (ii) comprises a low molecular weight, water-soluble polymer (e.g. hydroxypropyl cellulose, especially hydroxypropyl cellulose having a molecular weight of about 40,000 daltons) and one or more high molecular weight, water-soluble polymers (e.g. hydroxypropyl cellulose, especially two hydroxypropyl celluloses having molecular weights of about 140,000 daltons and 370,000 daltons. The film also preferably comprises a water-soluble polyethylene oxide, such as polyethylene oxide having a molecular weight of about 600,000 daltons.

The self-supporting, dissolvable, film may be a monolithic film where dexmedetomidine or a pharmaceutically acceptable salt thereof is substantially uniformally distributed throughout the polymeric film substrate. However, the self-supporting, dissolvable, film may preferably be a film comprising a polymeric film substrate onto the surface of which is deposited dexmedetomidine or a pharmaceutically acceptable salt thereof, especially when deposited as one or more discrete droplets which only partially cover the surface of the film substrate.

Dosage:

The dosing regimen employed in the present disclosure will depend on several factors, such as the severity or strength of the signs of the emergence of the agitation in a patient. Based on the severity/strength of the signs of the emergence of agitation (represented by physiological changes in the sympathetic nervous activities), in certain embodiments, the unit dose of an anti-agitation agent such as an alpha-2 adrenergic receptor agonist (e.g. dexmedetomidine or a pharmaceutically acceptable salt thereof) may vary in a range from about 3 micrograms to about 250 micrograms.

Thus, in one aspect, the amount of dexmedetomidine or a pharmaceutically acceptable salt thereof in a unit dose may be about 3 micrograms to 300 micrograms, about 3 micrograms to 250 micrograms, about 5 micrograms to 200 micrograms, about 5 micrograms to 180 micrograms, about 5 micrograms to 150 micrograms, about 5 micrograms to 120 micrograms, about 5 micrograms to 100 micrograms or about 10 micrograms to 50 micrograms. Specifically, the amount of dexmedetomidine or a pharmaceutically acceptable salt thereof in a unit dose may be about 5 micrograms, about 10 micrograms, about 15 micrograms, about 20 micrograms, about 25 micrograms, about 30 micrograms, about 35 micrograms, about 40 micrograms, about 45 micrograms, about 50 micrograms, about 55 micrograms, about 60 micrograms, about 65 micrograms, about 70 micrograms, about 75 micrograms, about 80 micrograms, about 85 micrograms, about 90 micrograms, about 95 micrograms, about 100 micrograms, about 110 micrograms, about 120 micrograms, about 130 micrograms, about 140 micrograms, about 150 micrograms, about 160 micrograms, about 170 micrograms, about 180 micrograms, about 190 micrograms, or about 200 micrograms.

In another aspect, the present disclosure provides a method of preventing the emergence of agitation in a subject identified by measuring one or more physiological signals of sympathetic nervous system activity as about to have an agitation episode, comprising administering to said subject an effective amount of dexmedetomidine or a pharmaceutically acceptable salt thereof at a dosage that does not cause significant sedation. In some embodiments, the unit dose of dexmedetomidine or a pharmaceutically acceptable salt thereof may be ranging from about 3 micrograms to about 300 micrograms, about 3 micrograms to about 270 micrograms, about 3 micrograms to about 250 micrograms, about 3 micrograms to about 240 micrograms, about 3 micrograms to about 200 micrograms, about 3 micrograms to about 180 micrograms, about 3 micrograms to about 150 micrograms, about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about 5 micrograms to about 15 micrograms, about 5 micrograms to about 10 micrograms, less than 10 micrograms (e.g. about 5, 6, 7, 8, or 9 micrograms), about 10 micrograms, about 12 micrograms, about 14 micrograms, about 15 micrograms, about 16 micrograms, about 18 micrograms, about 20 micrograms, about 30 micrograms, about 50 micrograms).

In a further aspect, the present disclosure provides a method of preventing the emergence of agitation in a subject identified by measuring one or more physiological signals of sympathetic nervous system activity as about to have an agitation episode, comprising administering to said subject an effective amount of dexmedetomidine or a pharmaceutically acceptable salt thereof at a dosage of from about 0.05 micrograms/kg weight of subject to about 3 micrograms/kg weight of subject. Examples of suitable dosages include: about 0.1 micrograms/kg to about 2.5 micrograms/kg, about 0.1 micrograms/kg to about 2 micrograms/kg, about 0.1 micrograms/kg to about 1.5 micrograms/kg, about 0.1 micrograms/kg to about 1 micrograms/kg, about 0.1 micrograms/kg to about 0.5 micrograms/kg, about 0.1 micrograms/kg to about 0.4 micrograms/kg, about 0.1 micrograms/kg to about 0.3 micrograms/kg, about 0.1 micrograms/kg to about 0.2 micrograms/kg, about 0.07 micrograms/kg, about 0.05 micrograms/kg, about 0.1 micrograms/kg, about 0.2 micrograms/kg, about 0.3 micrograms/kg, about 0.4 micrograms/kg, about 0.5 micrograms/kg, about 0.6 micrograms/kg, about 0.7 micrograms/kg, about 0.8 micrograms/kg, about 0.9 micrograms/kg, about 1.0 micrograms/kg, about 1.1 micrograms/kg, about 1.2 micrograms/kg, about 1.3 micrograms/kg, about 1.4 micrograms/kg, about 1.5 micrograms/kg.

The dose administration frequency may vary from one to more than one times a day depending upon the strength/severity of the physiological signals arising due to change in sympathetic nervous activity.

In yet other aspect, the present disclosure provides a method of preventing the emergence of agitation in a schizophrenic subject identified by measuring one or more physiological signals of sympathetic nervous system activity as about to have an agitation episode, comprising administering to said subject an effective amount of dexmedetomidine or a pharmaceutically acceptable salt thereof at a dosage that does not cause significant sedation. In some embodiments, the unit dose of dexmedetomidine or a pharmaceutically acceptable salt thereof may be ranging from about 3 micrograms to about 300 micrograms, about 3 micrograms to about 250 micrograms, about 3 micrograms to about 200 micrograms, about 3 micrograms to about 180 micrograms, about 3 micrograms to about 150 micrograms, about 5 micrograms to about 100 micrograms, about 5 micrograms to about 90 micrograms, about 5 micrograms to about 85 micrograms, about 5 micrograms to about 80 micrograms, about 5 micrograms to about 75 micrograms, about 5 micrograms to about 70 micrograms, about 5 micrograms to about 65 micrograms, about 5 micrograms to about 60 micrograms, about 5 micrograms to about 55 micrograms, about 5 micrograms to about 50 micrograms, about 5 micrograms to about 45 micrograms, about 5 micrograms to about 40 micrograms, about 5 micrograms to about 35 micrograms, about 5 micrograms to about 30 micrograms, about 5 micrograms to about 25 micrograms, about 5 micrograms to about 20 micrograms, about 5 micrograms to about 15 micrograms, about 5 micrograms to about 10 micrograms, less than 10 micrograms (e.g. about 5, 6, 7, 8, or 9 micrograms). In some embodiments, the unit dose of dexmedetomidine or a pharmaceutically acceptable salt thereof is about 10 micrograms, about 12 micrograms, about 14 micrograms, about 15 micrograms, about 16 micrograms, about 18 micrograms, about 20 micrograms, about 30 micrograms, about 50 micrograms, about 60 micrograms, about 70 micrograms, about 80 micrograms, about 90 micrograms, about 100 micrograms, about 110 micrograms, about 120 micrograms, about 130 micrograms, about 140 micrograms, about 150 micrograms, about 160 micrograms, about 170 micrograms, about 180 micrograms, about 190 micrograms, about 200 micrograms, about 210 micrograms, about 220 micrograms.

### EXAMPLE EMBODIMENTS

Embodiment 1. A method of selecting a patient for signs of emergence of agitation, comprising:
(a) placing or mounting an automated monitoring device on the patient's skin surface;
(b) monitoring one or more physiological signals of sympathetic nervous system activity in the patient with the said device;
(c) identifying a patient suitable for a therapy based on the assessment of the parameters of physiological signals of sympathetic nervous system activity monitored by the said device; and
(d) selecting a patient with increased sympathetic nervous system activity based on one or more physiological signals.

Embodiment 2. A method of preventing signs of emergence of agitation in a patient, comprising:
(a) placing or mounting an automated monitoring device on the patient's skin surface;
(b) monitoring one or more physiological signals of sympathetic nervous system activity in the patient with the said device;
(c) identifying a patient suitable for a therapy based on the assessment of the parameters of physiological signals of sympathetic nervous system activity, monitored by the said device;
(d) selecting a patient with increased sympathetic nervous system activity based on the physiological signals; and
(e) administering an anti-agitation agent to reduce the sympathetic nervous system activity in said patient.

Embodiment 3. A method of treating signs of emergence of agitation in a patient, comprising:
(a) placing or mounting an automated monitoring device on the patient's skin surface;
(b) monitoring one or more physiological signals of sympathetic nervous system activity in the patient with the help of said device;
(c) identifying a patient suitable for a therapy based on the assessment of the parameters of physiological signals of sympathetic nervous system activity, monitored by the said device;
(d) selecting a patient with increased sympathetic nervous system activity based on the physiological signals; and
(e) administering an anti-agitation agent to reduce the sympathetic nervous system activity in said patient.

Embodiment 4. The method according to any one of Embodiments 1-3, wherein the said automated monitoring device is a wearable device and remain in contact with patient's body.

Embodiment 5. The method according to any one of Embodiments 1-4, wherein the automated monitoring device detects changes in physiological signals related to sympathetic nervous system activity.

Embodiment 6. The method according to Embodiment 5, wherein the change in physiological signals related to sympathetic nervous system activity refers to an increase in the activity of sympathetic nervous system parameters.

Embodiment 7. The method according to Embodiment 5, wherein the physiological signals related sympathetic nervous system activity are selected from one or more of the following: change in skin conductance (GSR); electrodermal activity (EDA), temperature variability (skin temperature), electromyography (EMG) levels, heart rate variability such as resting EEG, ECG; actigraphy/polysomnography; cognitive assessments such as pupil size; secretion of salivary amylase; blood pressure; pulse rate; respiratory rate; level of oxygen in the blood and any other signal related to sympathetic nervous system activity.

Embodiment 8. The method according to any one of Embodiments 1-7, wherein the automated device sends signal data related to sympathetic nervous system activity of a patient to a remotely situated apparatus that is monitored by a caregiver.

Embodiment 9. The method according to any one of Embodiments 1-8, wherein the device worn by the patient sends a signal to a caregiver through substantially continuous data transfer technology (e.g., bluetooth or other transmission technology).

Embodiment 10. The method according to any one of Embodiments 1-9, wherein a caregiver becomes aware of a change in sympathetic nervous system activity and responds by administering a sympathetic nervous system activity reducing agent to prevent agitation from occurring.

Embodiment 11. The method according to any one of Embodiments 1-10, wherein the anti-agitation agent is an alpha-2 adrenergic receptor agonist selected from the group consisting of clonidine, guanfacine, guanabenz, guanoxabenz, guanethidine, xylazine, tizanidine, medetomidine, dexmedetomidine, methyldopa, methylnorepinephrine, fadolmidine, iodoclonidine, apraclonidine, detomidine, lofexidine, amitraz, mivazerol, azepexol, talipexol, rilmenidine, naphazoline, oxymetazoline, xylometazoline, tetrahydrozoline, tramazoline, talipexole, romifidine, propylhexedrine, norfenefrine, octopamine, moxonidine, lidamidine, tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof and preferably dexmedetomidine and or a pharmaceutically acceptable salt thereof.

Embodiment 12. The method according to Embodiment 11, wherein said dexmedetomidine or a pharmaceutically acceptable salt thereof is administered orally, buccally, trans-mucosally, sublingually or parenterally, and preferably by the sublingual route.

Embodiment 13. The method according to Embodiment 12, wherein the sublingual dosage form is selected from the group consisting of a film, wafer, patch, lozenge, gel, spray, tablet and liquid drops.

Embodiment 14. The method according to Embodiment 11 or 12, wherein said dexmedetomidine or a pharmaceutically acceptable salt thereof is administered at a unit dose in the range of about 3 micrograms to about 300 micrograms, about 3 micrograms to about 250 micrograms and preferably in dose range from about 5 micrograms to about 200 micrograms, more preferably about 5 micrograms to about 180 micrograms.

Embodiment 15. The method according to any one of Embodiments 1-14, wherein the patient is suffering from a neuropsychiatric disease, neurodegenerative disease or other nervous system related disease.

Embodiment 16. The method according to Embodiment 15, wherein said neuropsychiatric disease is selected from the group consisting of schizophrenia, bipolar disorder, bipolar mania, delirium, major depressive disorders and depression.

Embodiment 17. The method according to Embodiment 15, wherein said neurodegenerative disease is selected from the group consisting of Alzheimer's disease, frontotemporal dementia (FTD), dementia, dementia with Lewy bodies (DLB), post-traumatic stress disorder, Parkinson's disease, vascular dementia, vascular cognitive impairment, Huntington's disease, multiple sclerosis, Creutzfeldt-Jakob disease, multiple system atrophy, traumatic brain injury and progressive supranuclear palsy.

Embodiment 18. A method of preventing signs of emergence of agitation in patients with Schizophrenia comprising:
(a) placing or mounting an automated monitoring device on the patient's skin surface;
(b) monitoring one or more physiological signals of sympathetic nervous system activity in the patient with the help of said device;
(c) identifying a patient suitable for a therapy based on the assessment of the parameters of physiological signals of sympathetic nervous system activity, monitored by the said device;
(d) selecting a patient with increased sympathetic nervous system activity based on the physiological signals; and,
(e) administering an alpha-2 adrenergic receptor agonist to reduce the sympathetic nervous system activity in said patient.

Embodiment 19. A method of treating signs of emergence of agitation in patients with Schizophrenia comprising:
(a) placing or mounting an automated monitoring device on the patient's skin surface;
(b) monitoring one or more physiological signals of sympathetic nervous system activity in the patient with the help of said device;
(c) identifying a patient suitable for a therapy based on the assessment of the parameters of physiological signals of sympathetic nervous system activity, monitored by the said device;
(d) selecting a patient with increased sympathetic nervous system activity based on the physiological signals; and
(e) administering an alpha-2 adrenergic receptor agonist to reduce the sympathetic nervous system activity in said patient.

Embodiment 20. A method of preventing signs of emergence of agitation in patients with Delirium comprising:
(a) placing or mounting an automated monitoring device on the patient's skin surface;
(b) monitoring one or more physiological signals of sympathetic nervous system activity in the patient with the help of said device;
(c) identifying a patient suitable for a therapy based on the assessment of the parameters of physiological signals of sympathetic nervous system activity, monitored by the said device;
(d) selecting a patient with increased sympathetic nervous system activity based on the physiological signals; and
(e) administering an alpha-2 adrenergic receptor agonist to reduce the sympathetic nervous system activity in said patient.

Embodiment 21. A method of treating signs of emergence of agitation in patients with Delirium comprising:
(a) placing or mounting an automated monitoring device on the patient's skin surface;
(b) monitoring one or more physiological signals of sympathetic nervous system activity in the patient with the help of said device;
(c) identifying a patient suitable for a therapy based on the assessment of the parameters of physiological signals of sympathetic nervous system activity, monitored by the said device;
(d) selecting a patient with increased sympathetic nervous system activity based on the physiological signals; and
(e) administering an alpha-2 adrenergic receptor agonist to reduce the sympathetic nervous system activity in said patient.

Embodiment 22. A method of preventing signs of emergence of agitation in patient comprising:
(a) placing or mounting an automated monitoring device on the patient's skin surface;
(b) monitoring one or more physiological signals of sympathetic nervous system activity in the patient with the help of said device;
(c) identifying a patient suitable for a therapy based on the assessment of the parameters of physiological signals of sympathetic nervous system activity, monitored by the said device;
(d) selecting a patient with increased sympathetic nervous system activity based on the physiological signals; and
(e) administering dexmedetomidine or a pharmaceutically acceptable salt thereof to reduce the sympathetic nervous activities in said patient.

Embodiment 23. A method of treating signs of emergence of agitation in patients comprising:
(a) placing or mounting an automated monitoring device on the patient's skin surface;
(b) monitoring one or more physiological signals of sympathetic nervous system activity in the patient with the help of said device;
(c) identifying a patient suitable for a therapy based on the assessment of the parameters of physiological signals of sympathetic nervous system activity, monitored by the said device;
(d) selecting a patient with increased sympathetic nervous system activity based on the physiological signals; and
(e) administering dexmedetomidine or a pharmaceutically acceptable salt thereof to reduce the sympathetic nervous activities in said patient.

Embodiment 24. A method of preventing signs of emergence of agitation in patients comprising:
(a) placing or mounting an automated monitoring device on the patient's skin surface;
(b) monitoring one or more physiological signals of sympathetic nervous system activity in the patient with the help of said device;
(c) identifying a patient suitable for a therapy based on the assessment of the parameters of physiological signals of sympathetic nervous system activity, monitored by the said device;
(d) selecting a patient with increased sympathetic nervous system activity based on the physiological signals;
(e) determination of the intensity of the increased physiological signals of sympathetic nervous system activity in the selected patient, and
(f) administering dexmedetomidine or a pharmaceutically acceptable salt thereof to the patient to reduce the sympathetic nervous system activity, wherein the dose of the dexmedetomidine or a pharmaceutically acceptable salt thereof is selected based on the intensity of increased signals.

Embodiment 25. A method of treating signs of emergence of agitation in patients comprising:
(a) placing or mounting an automated monitoring device on the patient's skin surface;
(b) monitoring one or more physiological signals of sympathetic nervous system activity in the patient with the help of said device;
(c) identifying a patient suitable for a therapy based on the assessment of the parameters of physiological signals of sympathetic nervous system activity, monitored by the said device;
(d) selecting a patient with increased sympathetic nervous system activity based on the physiological signals;
(e) determination of the intensity of the increased signals of sympathetic nervous system activity in the selected patient; and
(f) administering dexmedetomidine or a pharmaceutically acceptable salt thereof to the patient to reduce the sympathetic nervous system activity, wherein the dose of the dexmedetomidine or a pharmaceutically acceptable salt thereof is selected based on the intensity of the strength of increased signals.

Embodiment 26: A method, comprising:
(a) receiving first physiological data of sympathetic nervous system activity;
(b) establishing a baseline value of at least one physiological parameter by training at least one machine learning model using the first physiological data;
(c) receiving, from a first monitoring device attached to a subject, second physiological data of sympathetic nervous system activity in the subject;
(d) analyzing, using the at least one machine learning model) and based on the baseline value of at least one physiological parameter, the second physiological data to predict an agitation episode of the subject; and
(e) sending, based on predicting the agitation episode of the subject, a signal to a second monitoring device to notify the second monitoring device of the prediction of the agitation episode in the subject such that treatment can be provided to the subject to decrease sympathetic nervous system activity in the subject.

Embodiment 27: The method of embodiment 26, wherein: the first monitoring device is a wearable device in contact with the subject.

Embodiment 28: The method of embodiment 26, wherein the second monitoring device is monitored by a caregiver of the subject.

Embodiment 29: The method of embodiment 26, wherein: the analyzing to predict the agitation episode includes determining a time period within which the agitation episode in the subject will occur.

Embodiment 30: The method of embodiment 26, wherein:

the analyzing to predict the agitation episode includes determining a degree of the agitation episode of the subject.

Embodiment 31: The method of embodiment 26, wherein:

the analyzing to predict the agitation episode includes:
comparing the second physiological data with the baseline value of at least one physiological parameter;
when the second physiological data exceeds a first threshold of the baseline value, the signal is a first signal, the treatments are first treatments;
when the second physiological data exceeds a second threshold of the baseline value, the signal is a second signal different from the first signal, the treatments are second treatments different from the first treatments.

Embodiment 32: The method of embodiment 26, wherein the receiving the second physiological data is during a first time period; the method further comprises:

receiving, during a second time period after the first time period, third physiological data of sympathetic nervous system activity in the subject; and
generating, based on the second physiological data and the third physiological data, a report of sympathetic nervous system activity in the subject to identify a pattern of a change of sympathetic nervous system activity in the subject.

Embodiment 33: The method of embodiment 26, wherein: the treatment includes administering an anti-agitation agent to the subject.

Embodiment 34: The method of embodiment 26, wherein:

the second physiological data of sympathetic nervous system activity include at least one of a change in electrodermal activity, heart rate variability, cognitive assessments such as pupil size, secretion of salivary amylase, blood pressure, pulse rate, respiratory rate, or level of oxygen in blood.

Embodiment 35: The method of embodiment 26, wherein:

the sympathetic nervous system activity is assessed by measuring any change in electrodermal activity or any change in electrodermal activity together with any change in resting electroencephalography.

Embodiment 36: The method of embodiment 26, further comprising:

receiving an indication associated with the agitation episode after sending the signal to the second monitoring device; and
further training the at least one machine learning model based on the indication.

Embodiment 37: The method of embodiment 26, further comprising:

receiving an indication associated with the agitation episode after sending the signal to the second monitoring device, the indication indicating at least one of (1) whether or not the agitation episode occurs, (2) when the agitation episode occurs, (3) a degree of the agitation episode, (4) a time period for which the agitation episode lasts, or (5) a symptom of the agitation episode; and further training the at least one machine learning model based on the indication.

Embodiment 38: The method of embodiment 26, wherein:

the at least one machine learning model includes at least one of a linear regression, logistic regression, a decision tree, a random forest, a neural network, a deep neural network, or a gradient boosting model.

Embodiment 39: The method of embodiment 26, wherein:

the at least one machine learning model is trained based on at least one of supervised learning, unsupervised learning, semi-supervised learning, or reinforcement learning.

Embodiment 39: The method of embodiment 26, wherein:

the analyzing to predict the agitation episode includes determining, based on a comparison between the second physiological data and the baseline value, a degree of the agitation episode of the subject.

Embodiment 40: An apparatus, comprising:

a memory; and
a processor operatively coupled to the memory, the processor configured to:
receive, from a first monitoring device attached to a subject, physiological data of sympathetic nervous system activity in the subject;
analyze, using at least one machine learning model, the physiological data to detect an anomaly from a reference pattern of sympathetic nervous system activity to determine a probability of an occurrence of an agitation episode in the subject; and
send a signal to a second monitoring device to notify the second monitoring device of the probability of the occurrence of the agitation episode in the subject such that treatment can be provided to the subject to decrease sympathetic nervous system activity in the subject.

Embodiment 41: The apparatus of embodiment 40, wherein:

the processor is configured to:
receive an indication associated with the agitation episode after sending the signal to the second monitoring device; and
further train the at least one machine learning model based on the indication.

Embodiment 42: The apparatus of embodiment 40, wherein:

the processor is configured to:
receive an indication associated with the agitation episode after sending the signal to the second monitoring device, the indication indicating one of (1) whether or not the agitation episode occurs, (2) when the agitation episode occurs, (3) a degree of the agitation episode, (4) a time period for which the agitation episode lasts, or (5) a symptom of the agitation episode; and further train the at least one machine learning model based on the indication.

Embodiment 43: A processor-readable non-transitory medium storing code representing instructions to be executed by a processor, the code comprising code to cause the processor to:

receive, from a first monitoring device attached to a subject, physiological data of sympathetic nervous system activity in the subject;
analyze, using at least one machine learning model, the physiological data to detect an anomaly from a reference pattern of sympathetic nervous system activity to determine a probability of an occurrence of an agitation episode of the subject; and
send a signal to a second monitoring device to notify the second monitoring device of the probability of the occurrence of the agitation episode of the subject such that treatment can be provided to the subject to decrease sympathetic nervous system activity in the subject.

Embodiment 44: The processor-readable non-transitory medium of embodiment 43, wherein the code comprises code to cause the processor to:

train, prior to analyzing using the at least one machine learning model, the at least one machine learning model based on training physiological data of sympathetic nervous system activity associated with a plurality of subjects, the at least one machine learning model including a plurality of physiological parameters as input, each physiological parameter from the plurality of physiological parameters associated with a weight from a plurality of weights of the machine learning model;
determine, based on the at least one machine learning model, the reference pattern of at least one physiological parameter from the plurality of physiological parameters.

Embodiment 45: The processor-readable non-transitory medium of embodiment 43, wherein the code comprises code to cause the processor to:

train, prior to analyzing using the at least one machine learning model, the at least one machine learning algorithm based on training physiological data of sympathetic nervous system activity associated with a plurality of subjects, the at least one machine learning model including a plurality of physiological parameters as input, each physiological parameter from the plurality of physiological parameters associated with a weight from a plurality of weights of the machine learning models;
determine, based on the at least one machine learning model, the reference pattern of at least one physiological parameter from the plurality of physiological parameters.
receive an indication associated with the agitation episode after sending the signal to the second monitoring device; and
further train, based on the indication, the at least one machine learning model to adjust the reference pattern of the at least one physiological parameter and a weight associated with the at least one physiological parameter.

Embodiment 46. The method, apparatus and processor-readable non-transitory medium storing code according to any one of Embodiments 1-45, wherein the automated monitoring device is a wearable device or a wearable sensor.

Embodiment 47. The method, apparatus and processor-readable non-transitory medium storing code according to any one of Embodiments 1-46, wherein the automated monitoring device detects change in physiological signals related to sympathetic nervous system activity.

Embodiment 48. The method, apparatus and processor-readable non-transitory medium storing code according to Embodiment 47, wherein the change in the physiological signals related to sympathetic nervous system activity refers to an increase in the activity of sympathetic nervous system parameters.

Embodiment 49. The method, apparatus and processor-readable non-transitory medium storing code according to Embodiment 48, wherein the physiological signals related to sympathetic nervous system activity comprises one or more of the following: change in Electrodermal activity (skin conductance); heart rate variability such as resting EEG, ECG; cognitive assessments such as pupil size; secretion of salivary amylase; blood pressure; pulse rate; respiratory rate, temperature variability, level of oxygen in the blood and any other signal related to sympathetic nervous system activity.

Embodiment 50. The method, apparatus and processor-readable non-transitory medium storing code according to Embodiment 47, wherein the change in the audio and motion signals related to sympathetic nervous system activity refers to an increase in the activity of sympathetic nervous system parameters.

Embodiment 51. The method, apparatus and processor-readable non-transitory medium storing code according to any one of Embodiments 1-50, wherein the automated monitoring device sends data of signals related to sympathetic nervous system activity in patients to a remotely situated apparatus which is monitored by a caregiver.

Embodiment 52. The method, apparatus and processor-readable non-transitory medium storing code according to any one of Embodiments 1-51, wherein the automated monitoring device sends a signal to a caregiver though Bluetooth or any other transmission-related technology.

Embodiment 53. The method, apparatus and processor-readable non-transitory medium storing code according to any one of Embodiments 1-52, wherein the caregiver becomes aware of the change in sympathetic nervous system activity and responds by administering a sympathetic nervous activities reducing amount of an anti-agitation agent, such as an alpha-2 adrenergic receptor agonist to prevent agitation from occurring.

Embodiment 54. The method, apparatus and processor-readable non-transitory medium storing code according to any one of Embodiments 1-53, wherein the anti-agitation agent is an alpha-2 adrenergic receptor agonist selected from the group consisting of clonidine, guanfacine, guanabenz, guanoxabenz, guanethidine, xylazine, tizanidine, medetomidine, dexmedetomidine, methyldopa, methylnorepinephrine, fadolmidine, iodoclonidine, apraclonidine, detomidine, lofexidine, amitraz, mivazerol, azepexol, talipexol, rilmenidine, naphazoline, oxymetazoline, xylometazoline, tetrahydrozoline, tramazoline, talipexole, romifidine, propylhexedrine, norfenefrine, octopamine, Moxonidine, Lidamidine, Tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof, and is preferably dexmedetomidine and or a pharmaceutically acceptable salt thereof.

Embodiment 55. The method, apparatus and processor-readable non-transitory medium storing code according to Embodiment 54, wherein said dexmedetomidine or a pharmaceutically acceptable salt thereof is administered orally, buccally, trans-mucosally, sublingually or parenterally and preferably sublingually.

Embodiment 56. The method, apparatus and processor-readable non-transitory medium storing code according to Embodiment 55, wherein the sublingual dosage form is selected from the group consisting of a film, wafer, patch, lozenge, gel, spray, tablet and liquid drops.

Embodiment 57. The method, apparatus and processor-readable non-transitory medium storing code according to any one of Embodiments 54-56, wherein said dexmedetomidine or a pharmaceutically acceptable salt thereof is administered at a dosage in the range of about 3 micrograms to about 300 micrograms, about 3 micrograms to about 250 micrograms and preferably in dose range from about 5 micrograms to about 200 micrograms and more preferably about 5 micrograms to about 180 micrograms.

Embodiments 58. The method, apparatus and processor-readable non-transitory medium storing code according to any one of Embodiments 1-57, wherein the patient is suffering from a neuropsychiatric disease, neurodegenerative disease or other nervous system related disease.

Embodiment 59. The method, apparatus and processor-readable non-transitory medium storing code according to Embodiment 58, wherein said patient is suffering from a neuropsychiatric disease selected from the group consisting of schizophrenia, bipolar disorder, bipolar mania, delirium, major depressive disorders and depression.

Embodiment 60. The method, apparatus and processor-readable non-transitory medium storing code according to Embodiment 58, wherein said patient is suffering from a neurodegenerative disease selected from the group consisting of Alzheimer's disease, frontotemporal dementia (FTD), dementia, dementia with Lewy bodies (DLB), post-traumatic stress disorder, Parkinson's disease, vascular dementia, vascular cognitive impairment, Huntington's disease, multiple sclerosis, Creutzfeldt-Jakob disease, multiple system atrophy, progressive supranuclear palsy, traumatic brain injury or other related neurodegenerative disease.

Embodiment 61. The method, apparatus and processor-readable non-transitory medium storing code according to Embodiment 59, wherein said patient is suffering from delirium.

Embodiment 62. The method, apparatus and processor-readable non-transitory medium storing code according to Embodiment 60, wherein said patient is suffering from dementia.

Embodiment 63. The method, apparatus and processor-readable non-transitory medium storing code according to any one of Embodiments 1-62, wherein the patient is suffering from opioid, substance (including cocaine, amphetamine) or alcohol withdrawal.

Embodiment 64. The method, apparatus and processor-readable non-transitory medium storing code according to any of the embodiment 1 to 60, wherein the additional signals of sympathetic nervous system activity include audio and motion.

The following Examples are intended to be illustrative, and not limiting. Thus, Example 1 is illustrative of a sublingual composition of dexmedetomidine hydrochloride for use in the present disclosure and its preparation.

### Example 1

TABLE 1
Dexmedetomidine deposited on the surface of a polymer matrix film composition:

| | | | |
|---|---|---|---|
| | Concentration | Concentration | |
| | g/100 g | g/100 g | |
| Ingredients | (10 μg film) | (20 μg film) | Function |

Drug-containing composition

| | | | |
|---|---|---|---|
| Dexmedetomidine | 0.135811 | 0.267271 | Active agent |
| hydrochloride | | | |
| Hydroxypropyl cellulose, HPC- | 0.301242 | 0.592835 | Film former |
| SSL (MW = 40,000) | | | |
| Hydroxypropyl cellulose | 0.301242 | 0.592835 | Film former |
| (MW = 140,000) | | | |
| FD&C Blue #1 Granular | 0.002222 | 0.004372 | Color |
| Ethyl Alcohol as a solvent | qs | qs | Solvent |

Polymer matrix composition

| | | | |
|---|---|---|---|
| Hydroxypropyl cellulose | 4.803166 | 4.768481 | Film former |
| (MW = 140,000) | | | |
| Hydroxypropyl cellulose, HPC- | 4.803166 | 4.768481 | Film former |
| SSL | | | |
| (MW = 40,000) | | | |
| Hydroxypropyl cellulose | 28.80907 | 28.60103 | Film former |
| (MW = 370,000) | | | |
| Fast Emerald Green Shade (NO. | 0.129037 | 0.128105 | Color |
| 06507) | | | |
| Sucralose, USP-NF Grade | 0.992595 | 0.985427 | Sweetener |
| Peppermint Oil, NF | 2.104301 | 2.089105 | Flavor |
| Polyethylene oxide | 57.61815 | 57.20206 | Film former & |
| Sentry Polyox WSR 205 LEO | | | Mucoadhesive |
| NF (MW = 600,000) | | | |
| Water as a solvent | qs | qs | Solvent |

(A) Process for the Preparation of Polymer Matrix

Polymer mixture: Polyethylene oxide and fast emerald green shade were mixed in water for at least 180 minutes at about 1400 rpm to about 2000 rpm. Sucralose, hydroxypropyl cellulose (molecular weight 140K), hydroxypropyl cellulose, HPC-SSL (molecular weight 40K) and hydroxypropyl cellulose (molecular weight 370K) were added and mixed for at least 120 minutes at about 1600 rpm to 2000 rpm. Peppermint Oil was added to water and the resultant dispersion was then added to the polymer mixture and mixed for at least 30 minutes. The resultant mixture was further mixed under vacuum (248 torr) for at least for 30 minutes at a speed of 350 rpm and at temperature of 22.9° C.

Coating station: A roll was placed on an unwind stand and the leading edge was thread through guide bars and coating bars. The silicone-coated side of the liner was placed faced up. A gap of 40 millimeters was maintained between the coating bars. The oven set point was adjusted to 70° C. and the final drying temperature was adjusted to 85° C.

Coating/drying process: The polymer mixture was poured onto the liner between the guide bars and the coating bars. The liner was pulled slowly through the coating bar at a constant speed by hand until no liquid was remained on the coating bars. The liner was cut to approximately 12-inch length hand sheets using a safety knife. Each hand sheet was placed on a drying board and was tapped on the corners to prevent curl during drying. The hand sheets were dried in the oven until the moisture content was less than 5% (approximately 30 minutes) and then removed from the drying board. The coating weights were checked against the acceptance criteria, and if met, the hand sheets were then stacked and placed in a 34 inch×40 inch foil bag that was lined with PET release liner.

(B) Process for the Preparation of Deposition Solution:

FDC blue was dissolved in ethyl alcohol for at least 180 minutes. Dexmedetomidine hydrochloride was added to the ethyl alcohol solution with continuous stirring for 10 minutes at about 400 rpm to about 800 rpm. Hydroxypropyl cellulose (40K) and hydroxypropyl cellulose (140K) were added to the mixture, and stirred for at least 30 minutes until all the materials were dissolved.

(C) Process for the Preparation of Micro-Deposited Matrix:

The deposition solution obtained in Step (B) above was filled into a pipette to the required volume (determined according to the specific drug product strength of the final product). An appropriate amount (1.5 microliters=approximately 5 micrograms) of the deposition solution were deposited (e.g. as droplets) onto the polymer matrix obtained in Step (A), and repeated to a total of 10 times (i.e. 10 deposits/droplets) with space between each deposit to prevent merging of the deposits/droplets and allow subsequent cutting of the film into individual drug-containing units. The film was initially die cut in individual units with dimensions of 22 mm×8.8 mm containing a single deposit of the drug-containing composition. The die cut micro-deposited matrixes were then dried in an oven for 70° C. for 10 minutes and further die cut into 10 units with each unit containing a single deposit of the drug-containing composition.

(D) Packaging:

Each defect-free unit was sealed individually into a foil pouch, which was then heat sealed. If the heat seal was acceptable the package was considered as an acceptable unit for commercial use.

Other unit strengths (e.g. 40 μg and 60 μg films) were similarly prepared by varying the concentrations of drug, polymers and colorant within the drug-containing composition. For example, the 40 μg and 60 μg films were prepared from drug-containing compositions containing, respectively, approximately 2×, and 3×, the amounts of drug, polymers and colorant that appear in the 20 μg drug-containing composition described in Table 1 above.

TABLE 2
Dexmedetomidine deposited on the surface of a polymer matrix film composition

| | | | | |
|---|---|---|---|---|
| | Concentration | Concentration | Concentration | |
| | mg/unit | mg/unit | mg/unit | |
| Ingredients | (80 μg film) | (120 μg film) | (180 μg film) | Function |

Drug-containing composition

| | | | | |
|---|---|---|---|---|
| Dexmedetomidine | 0.095 | 0.142 | 0.213 | Active agent |
| hydrochloride | | | | |
| Hydroxypropyl | 0.081 | 0.122 | 0.183 | Film former |
| cellulose, HPC-SSL | | | | |
| (MW = 40,000) | | | | |
| Hydroxypropyl | 0.081 | 0.122 | 0.183 | Film former |
| cellulose | | | | |
| (MW = 140,000) | | | | |
| FD&C Blue #1 | 0.001 | 0.001 | 0.002 | Color |
| Granular | | | | |
| Ethyl Alcohol as a | q.s | q.s. | q.s. | Solvent |
| solvent | | | | |

Polymer matrix composition

| | | | | |
|---|---|---|---|---|
| Hydroxypropyl | 0.627 | 0.627 | 0.627 | Film former |
| cellulose | | | | |
| (MW = 140,000) | | | | |
| Hydroxypropyl | 0.627 | 0.627 | 0.627 | Film former |
| cellulose, HPC-SSL | | | | |
| (MW = 40,000) | | | | |
| Hydroxypropyl | 3.763 | 3.763 | 3.763 | Film former |
| cellulose | | | | |
| (MW = 370,000) | | | | |
| Fast Emerald Green | 0.017 | 0.017 | 0.017 | Color |
| Shade (NO. 06507) | | | | |
| Sucralose, USP-NF | 0.130 | 0.130 | 0.130 | Sweetener |
| Grade | | | | |
| Peppermint Oil, NF | 0.275 | 0.275 | 0.275 | Flavor |
| Polyethylene oxide | 7.526 | 7.526 | 7.526 | Film former & |
| (Sentry Polyox WSR | | | | Mucoadhesive |
| 205 LEO NF) (MW = | | | | |
| 600,000) | | | | |
| Water as a solvent | qs | qs | qs | Solvent |

The formulations (80 μg, 120 μg and 180 μg) in table 2 were prepared using the same manufacturing process as described above for table 1.

### Example 2

Study to examine the safety and efficacy of a sublingual film delivery of dexmedetomidine hydrochloride for the treatment of acute agitation in Schizophrenia

This study is designed to examine the dose-related efficacy and tolerability of sublingual dexmedetomidine hydrochloride on clinical ratings and objective biomarkers of agitation, autonomic arousal and sedation in patients with schizophrenia. Outcome measures include a well-validated clinical measure of agitation (PANS S-EC), a clinical measure of sedation (ACES/RASS), and physiological measures of hyperarousal:

a. Skin Conductance Response

b. Heart Rate Variability

c. Measures of Sleep: Actigraphy/Polysomnogram (PSG)

d. Exploratory Resting Electroencephalogram (EEG) and PSG that will be used in conjunction with other psychophysiological outcome measures to develop a predictive biomarker model of efficacy.

Example Research Plan:

This study aims to examine the effects of a sublingual film formulation of dexmedetomidine hydrochloride in patients with schizophrenia versus placebo on a range of symptom-related outcomes and more proximal potential biomarkers of efficacy.

In this study, the initial dose of sublingual dexmedetomidine hydrochloride will be 100 micrograms (μg) with the desired endpoint being the attainment of arousable sedation that can be reversed temporarily by verbal stimulation. If the end point is not reached and the drug is well-tolerated (as defined below), an additional 60 μg dose will be administered after 60 minutes or repeated 20 μg doses at intervals of approximately 60 minutes up to a total of 3 extra 20 μg doses (OR total of 160 μg/day).

Participants will be evaluated, as described below, after each dose, and once the participant is sedated, but able to respond to verbal stimulation, no more doses will be administered.

The plan is to run a cohort of about up to 20 subjects. An initial dose of dexmedetomidine hydrochloride will be 100 μg as described above. After at least 6 subjects are run, if the desired outcome is not achieved in at least ⅔ participants, a second dose level cohort may be initiated. In this second cohort, based upon the safety and tolerability observed with the first cohort, the initial dose of dexmedetomidine hydrochloride will be 120-160 μg sublingual with similar incremental dosing by 20 μg or a single 60 μg dose with the desired endpoint being one of the following 1) the attainment of arousable sedation that can be reversed temporarily by verbal stimulation, 2) attaining a ≥50% reduction of PEC total score; 3) ACES rating of 5, 6, or 7 (mild, moderate or marked calmness) without sedation (as measured by ACES rating of 8 or 9, deep or unarousable sleep). The total maximum dose of dexmedetomidine hydrochloride administered to a subject on a test day will not exceed 180 mcg. As such, if a starting dose of 160 μg is used, then only one additional 20 μg dose of dexmedetomidine hydrochloride will be administered on that test day. As in the first cohort, if the end point is not reached and the drug is well tolerated (as defined below), 20 μg will be repeated every 60 minutes up to a total of 3 additional 20 μg doses or a single 60 μg dose will be administered up to 180 μg per day. Once the participant is sedated but able to respond to verbal stimulation, no more doses will be administered.

The participants will be monitored by the site personnel, and vital signs including blood pressure, heart rate, and level of oxygen in the blood will be measured and recorded at regular intervals (approximately every 15 minutes) up to 2 hours after the last dose. In case subjects experience changes in vital signs that do not return to baseline by the 2-hour post-last dose timepoint, vital signs will also be collected hourly for up to 6 hours to determine if there is any delayed effect on vital signs. Based on the available data, we do not anticipate any changes this far out after dosing. However, longer duration of monitoring may be continued if deemed clinically necessary. Electrocardiography (EKG) will be performed at screening, baseline (pre-dose), post-dose, as well as the day after.

### Example Primary Outcome Measures:

1) PANSS-EC Change from Baseline: The Positive and Negative Syndrome Scale-Excited Component (PANSS-EC) comprises 5 items associated with agitation: poor impulse control, tension, hostility, uncooperativeness, and excitement; each scored 1 (min) to 7 (max). The PANSS-EC is the sum of these 5 subscales and ranges from 5 to 35. PANSS will be measured at screening, on Day 1 at baseline (pre-dose) and every 30 minutes post-dose and on Day 2.

2) Psychophysiological measures of arousal, such as skin conductance response (SCR), heart rate variability, and blood pressure: assessed at baseline and several times after drug administration.

3) Other psychometric measures of agitation will include:

a. ACES (Agitation-Calmness Scale): Designed to assess the clinical levels of calmness and sedation. This is a 9-point scale that differentiates between agitation, calmness, and sleep states Scores range from 1 (marked agitation) to 9 (unarousable).

b. RASS (Richmond Agitation Sedation Scale) change from baseline: The RASS is a 10-level rating scale ranging from “Combative” (+4) to “unarousable” (−5). ACES/RASS scores will be measured at screening, on Day 1 at baseline (pre-dose) and about every 30 minutes post-dose and on Day 2.

### Example Secondary Outcome Measures:

1) BARS (Behavioral Activity Rating Scale): Change from baseline ranging from 1 to 7 where: 1=difficult or unable to rouse, 2=asleep but responds normally to verbal or physical contact, 3=drowsy, appears sedated, 4=quiet, and awake (normal level of activity), 5=signs of overt (physical or verbal) activity, calms down with instructions, 6=extremely or continuously active, not requiring restraint, 7=violent, requires restraint.

2) Clinical Global Impressions-Improvement Scale (CGI-I) After Drug Administration CGI-I scores range from 1 to 7:0=not assessed (missing), 1=very much improved, 2=much improved, 3=minimally improved, 4=no change, 5=minimally worse, 6=much worse, 7=very much worse.

3) Determine any adverse effects on blood pressure, heart rate, or respiratory drive occurring before or coincident with the achievement of the aforementioned level of sedation.

### Example Tolerability Guidelines:

Dosing will be stopped for a subject at any time if any of the following occurs:

1) >30 mm Hg decrease in supine systolic or diastolic blood pressure

2) isolated drop in systolic BP<100 mmHg (The study will exclude patients with a resting supine systolic BP<110 mm Hg)

3) isolated drop diastolic BP<60 mmHg (the study will exclude patients with a resting diastolic BP<70 mmHg)

4) heart rate below 50 beats per minute (The study will exclude patients with a resting heart rate of <60 beats/minute)

5) Attainment of ACES end point rating of 5, 6, or 7 (mild, moderate or marked calmness)

6) Attainment of a RASS of −2 post dose.

Whenever the above stopping criteria is met, whether because of ACES/RASS score, BP or HR, we will continue to monitor the participant's vital signs every 15 minutes until the participant has reached their baseline parameters or, in the judgment of the principal investigator, the participant has reached a stable and acceptable level of blood pressure and heart rate. Sedation will be assessed every 30 minutes until the participant has reached a stable and acceptable level of arousal in the judgment of the principal investigator. Each subsequent starting dose will be determined based on a review of the results of the previous dosing cohorts by a team comprised of representatives from the sponsor and the site. This review will occur approximately 1 to 4 weeks after completion of the previous cohort.

Adverse events (AEs), including serious adverse events (SAEs), will be assessed, recorded, and reported in accordance with FDA guidance. Should any SAE occur the study will be stopped until a cause for the SAE has been determined.

Questionnaires/Behavioral Outcome Measures

In addition to the outcome measures as described above, sleep will be assessed using the Pittsburgh Sleep Quality Index and the Stanford Sleepiness Scale. A self-administered tool for assessing alertness will also be given to participants to complete on Study Days 0-2.

Psychophysiological Outcome Measures

Skin Conductance Response (SCR):

SCR is one of the fastest-responding measures of stress response and arousal. Along with changes in heart rate, it has been found to be one of the most robust and non-invasive physiological measures of autonomic nervous system activity. Studies have examined SCR to neutral tones in schizophrenia and reported hyperreactivity. Further, several authors have reported lower SCR in schizophrenia as well as a correlation with symptom severity and time to relapse.

SCR will be recorded using the Biopac MP150 system, using 11-mm inner diameter Ag/AgCl electrodes filled with isotonic electrode paste. The electrodes will be attached to the middle phalanges of the fourth and fifth fingers of the non-dominant hand. SCR waveforms will be analyzed with Acknowledge software or MATLAB, with base-to-peak difference assessed for the largest deflection in the window one to four seconds following stimulus onset.

Resting EEG:

Several pre-clinical and some clinical studies have examined EEG outcomes associated with dexmedetomidine effects. However, no studies have utilized the change in resting EEG pattern to distinguish clinical reduction of agitation versus sedation. A theoretical approach will be utilized to identify EEG patterns associated with reduction in agitation scores. EEG data will also be included in a model with skin conductance and actigraphy/polysomnography to provide the best fit for biomarkers related to the effects of dexmedetomidine.

The EEG will be recorded from an electrode cap containing a montage of scalp electrodes ranging from 3 to 128. The cap includes one ground electrode placed above the forehead, and a set of linked reference electrodes, one placed on each ear lobe.

Vertical and horizontal electro-oculograms (VEOG and HEOG) will be recorded and used to correct EEG data for eye blink and eye movement. EEG activity (e.g. spectral power, topographic microstate, and interelectrode coherence) during wakeful rest has been shown to be sensitive to psychosis/arousal. Recordings will therefore be obtained during up to three minutes of closed-eye resting EEG. Subjects will be told to relax with eyes closed for the session and told to remain as still as possible to minimize movement artifacts in the EEG.

PSG:

Measurements will be taken with a dry system (Cognionix) or with TEMEC or COMPUMEDICS system with EEG with scalp electrodes, electromyography with electrodes placed on the skin of the chin and limbs, electrocardiography with electrodes placed on the torso and limbs and electrooculography, and/or with electrodes on the forehead and temples. Pulse oximetry will be used to measure oxygen saturation during PSG. Orinasal thermal sensor and nasal air pressure transducer will be used to measure airflow, and respiratory effort will be measured with inductance plethysmography.

Heart Rate Variability:

Heart rate variability (HRV) is a measure of the variability in time intervals between heart beats and is sensitive to sympathetic activity as well as worsening of psychosis/agitation. In order to measure HRV, electrodes will be placed on the subject's chest and limbs.

Actigraphy:

Actigraphy is a non-invasive measure of rest/activity cycles in human beings. Subjects will wear a small actigraphy device, about the size of a wrist watch, strapped to the arm. This device will measure gross motor movement, step count, periods of sitting/laying, and physical activity. Subjects may be asked to wear the actigraphy device from the time of admission until discharge.

### Example Specific Procedures by Visit:

### Example Screening

The study will begin with 1-2 screening visits that will take place at a hospital. If the Principle Investigator deems it necessary, the subject maybe admitted to the hospital to finish the screening visit.

Approximately 40 participants are expected to be screened in this study for a target of approximately 20 completing the study in up to 4 cohorts. Participants may be included in more than one cohort. If more cohorts are needed to identify the appropriate dose, an amendment will be submitted.

The following tests and procedures will be performed to determine eligibility:

Review of medical, surgical and psychiatric history

Review of current and past medications (prescription, non-prescription, and dietary supplements)

Physical examination

Measurement of height, weight, and vital signs (blood pressure, heart rate, and temperature)

Measurement of orthostatic blood pressure

Completion of questionnaires related to current diagnosis and suicidal thoughts/behaviors (i.e., Columbia-Suicide Severity Rating Scale [CSSRS])

Cognitive testing to test memory and attention may be administered

Resting EEG

Skin Conductance Response at screening.

Electrocardiogram

Laboratory tests including:

Routine complete blood count, chemistry panel, TSH, tests for hepatitis B, C and HIV/AIDS

Pregnancy testing for women who can become pregnant. In some instances, the result of the pregnancy test must be negative to qualify to participate in this study

Routine urine analysis

Alcohol breathalyzer

Urine testing for drug abuse

Day 0 (it is possible that this may be combined with either Screening or Day 1 for participant convenience):

If found to be eligible after the screening visits (no more than 60 days prior to baseline), study participants will be scheduled for up to 3-day in-patient stay at the hospital for the purposes of study participation. Day 0 (Admission day): They will be asked to provide a urine sample to test for illicit substances. If the urine test result is positive, the Principle Investigator will be notified and participation in the study may be postponed or terminated. Females will also be tested for pregnancy. If the result of the urine pregnancy test is positive, study participation will be cancelled. Participants will be expected to arrive in the morning, and hospital staff will conduct a physical examination, interview, collect blood to perform standard metabolic laboratory tests and will administer an electrocardiogram. Subjects will be acclimatized to the in-patient unit and study procedures. Baseline psychophysiological assessments, including SCR, HRV and resting EEG and clinical rating scales, may be completed. Questionnaires related to current suicidal thoughts/behaviors (i.e., Columbia-Suicide Severity Rating Scale [CSSRS]) will be administered.

Day 1:

Baseline assessments including vital signs, psychophysiological outcome measures (including resting EEG, SCR, EKG) and behavioral assessments (including PANSS, ACES, RASS) will be followed by IV-line placement and study drug administration. Prior to administration of the study drug, subjects, in some instances, must demonstrate a score of ≥14 on the PANSS-EC. If subjects do not score ≥14 on the PANSS-EC within 15 minutes of dosing, the dosing will not initiate. Vital signs will be assessed frequently (15 minutes intervals or more frequently as needed) post dose. Participants will be monitored for at least up to 2 hours post-dose administration or until vital signs are stable and the level of sedation is acceptable. To summarize, before the administration of study medication (dexmedetomidine hydrochloride or placebo), the following procedures will take place:

Vital Signs (blood pressure, pulse, and level of oxygen in the blood)

Measurement of orthostatic blood pressure

Psychophysiological outcome measures

IV placement

Behavioral/Clinical outcome measures

Blood sample for PK analysis and neurochemical assays

The assigned study drug will then be administered sublingually by the study staff followed by:

Vital signs (blood pressure, pulse, and level of oxygen in the blood) taken every 15 minutes up to 2 hours after the last dose.

Measurement of orthostatic blood pressure prior to allowing the subject to ambulate

Psychophysiological outcome measures

Behavioral/clinical outcome measures every 30 minutes

Blood samples for PK analysis and neurochemical assays at approximately time 0, +30, +60, and +120 minutes after each dose. If the +60/+120 time-points for a dose coincide with a different time-point (example “0” timepoint) for a subsequent dose, only a single blood sample may be drawn. In addition, blood samples will be drawn approximately 4 and 8 hours post-last dose. Additional blood samples for PK/assays and safety laboratory tests will be drawn on day 2.

After achieving the desired level of sedation (as determined by the ACES/RASS), any other tolerability criteria (blood pressure or pulse changes) or approximately 2 hours after the last dose, subjects will undergo the following tests:

Electrocardiogram (ECG)

Post psychophysiological outcome measures (per Principle Investigator discretion)

In the case that subjects experience changes in vital signs that do not return to baseline by the 2-hour post-last dose time-point, vital signs (blood pressure, pulse, and level of oxygen in the blood) will also be taken hourly for up to 6 hours after the last dose, or further if deemed clinically necessary

ACES/RASS and clinical assessment for acceptable level of sedation

Overnight sleep assessment: PSQI and PSG/Actigraphy

Day 2

Subjects will meet with a study personnel to assess for any adverse events or side effects from the study drug. The following procedures will take place before discharge from the research site:

Vital signs

Measurement of orthostatic blood pressure

ECG

Behavioral/clinical outcome measures

Safety laboratory tests

Blood draw for PK/assays

Administration of the C-SSRS

Following the procedures on Day 2, participants will be discharged if deemed medically acceptable.

### Example Follow-up

There will be a follow-up post-procedure phone call within 1 week to assess for the following:

Participants can be asked about any medications taken since departure from the hospital

The C-SSRS can be administered

Adverse events can be assessed: subjects will be asked general questions about their well-being since departure from the hospital. Questions regarding the occurrence of specific adverse events will not be asked unless information is first volunteered by the subject.

If needed participants can be invited back for an in-person safety and follow-up evaluation.

If a research subject is found to be acutely suicidal, he or she may be taken to a psychiatric emergency room or involuntarily admitted to the hospital for treatment of the suicidal ideation. Acutely suicidal patients will not be allowed to continue in the study and will need to be re-screened at a later date if they are still interested in participating.

TABLE 3
Schedule of activities overview

| Activity | Screen | Day 0 | Day 1 | Day 2 | Follow-up |
|---|---|---|---|---|---|
| ICF | X | | | | |
| Medical History | X | X | X | | |
| Demographics | X | | | | |
| Psychiatric | X | X | X | | |
| Evaluation | | | | | |
| SCID | X | | | | |
| I/E criteria | X | | | | |
| Randomization | | X | | | |
| Safety Labs | X | X | X | X | |
| Physical Exam | X | X | | | |
| Vital Signs | X | X | X* | X | |
| Orthostatic Blood | X | X | X | X | |
| Pressure | | | | | |
| ECG | X | X | X | X | |
| PANSS | X | | X* | | |
| RASS | X | | X* | X | |
| Skin Conductance | X | | X* | | |
| Resting EEG | X | | X* | | |
| Study Drug | | | X | | |
| PK sampling, | | | X* | | |
| sampling for | | | | | |
| neurochemical | | | | | |
| assays | | | | | |
| Concomitant | X | | X | X | X |
| Medications | | | | | |
| Adverse Events | X | | X | X | X |
| ACES | X | | X* | X | |
| BARS | X | | X* | X | |

*several times at baseline (pre-dose) and post-dose on test day

To take orthostatic blood pressure, research staff can require the subject to lie down for 5 minutes. After 5 minutes, research staff will measure blood pressure and pulse rate. The subject can then be asked to stand up. The blood pressure and pulse rate measurements can be taken again after the subject has been standing for 1 and 3 minutes. A drop in BP of ≥20 mm Hg, or in diastolic BP of ≥10 mm Hg, or if the subject is experiencing light headedness or dizziness, research staff can initiate fall precautions for the subject.

Number of Subjects:

Subjects with a diagnosis of Schizophrenia Spectrum Disorder will be recruited. The study aims to enroll patients with psychosis who do not currently require an in-patient hospitalization. Target sample size is 20 and target enrolment is 40.

### Example Inclusion Criteria:

1. Ability to give informed consent.

2. Male or female between 18 and 65 years of age, inclusive.

3. According to DSM-V, meet criteria for schizophrenia or schizoaffective disorder.

4. In the opinion of the Principal Investigator or designee, sufficiently physically healthy to receive a sublingual dose of dexmedetomidine hydrochloride sufficient to cause sedation temporarily arousable by verbal stimulation.

5. Patients who are in good general health prior to study participation as determined by a detailed medical history, physical examination, 12-lead ECG, blood chemistry profile, hematology, urinalysis, and in the opinion of the Principal Investigator.

6. Female participants, if of child-bearing potential (women who have not yet attained documented menopause will be considered of child-bearing potential unless we have documentation that they have undergone a hysterectomy) and sexually active, who agree to use a medically acceptable and effective birth control method for 30 days before and after the study. Male participants, if sexually active with a partner of child-bearing potential, who agree to use a medically acceptable and effective birth control method throughout the study and for three months following the end of the study. Medically acceptable methods of contraception that may be used by the participant and/or his/her partner include abstinence, birth control pills or patches, diaphragm with spermicide, intrauterine device (IUD), condom with foam or spermicide, vaginal spermicidal suppository, surgical sterilization and progestin implant or injection. Prohibited methods include: the rhythm method, withdrawal, condoms alone, or diaphragm alone.

7. At baseline (15 minutes prior to treatment), PANSS-EC score of ≥14.

### Example Exclusion Criteria

1. Patients with agitation caused by acute intoxication.

2. Positive identification of non-prescription drugs at baseline

3. Patients treated with benzodiazepines or other hypnotics or oral or short-acting intramuscular antipsychotics for agitation within 6 hours prior to study drug administration. If the patient requires a PRN benzodiazepine for agitation, we will not proceed with the test day.

4. Focal neurological deficits or clinically significant neurological disorder.

5. Presence of clinically significant or unstable medical illnesses that in the opinion of the Principal Investigator or designee makes the patient unsuitable for participation in this study.

6. Acute increased risk of suicide in the judgment of the Principal Investigator or designee.

7. Significant clinical laboratory abnormalities (including positivity for Hep B, Hep C, HIV) unless treated to remission status.

8. Drug or alcohol use disorder within the last 6 months in the opinion of the Principal Investigator or designee (excluding nicotine).

9. Presence of any of the following cardiovascular comorbidities: advanced heart block (second-degree or above atrioventricular block without pacemaker), diagnosis of sick sinus syndrome, hypovolemia, insulin-dependent diabetes mellitus, chronic hypertension not adequately controlled by antihypertensive medications, history of syncope or other syncopal attacks, current evidence of orthostatic hypotension, have a resting heart rate of <60 beats per minutes or systolic blood pressure <110 mmHg or diastolic BP<70 mmHg, have evidence of a clinically significant 12 lead ECG abnormality.

10. Presence of Moderate-to-severe hepatic impairment (Pugh-Childs score ≥7).

11. Treatment with alpha-1 noradrenergic blocking drugs as well as alpha-2 agonist medications such as clonidine and guanfacine

12. Pregnant and lactating women

13. History of allergic reactions to dexmedetomidine or known allergy to dexmedetomidine.

### Example Eligibility Criteria:

Subjects may first undergo a phone screen to initially determine eligibility. Information collected during the phone screen will only be used in the event that the subject continues to participate in the study.

After determining initial eligibility, research staff will provide a brief description of the research and the subject will present to the clinic for the screening procedures described above. Once all screening procedures have been collected, research staff, as well as the Principal Investigator, will review all relevant information and determine, based on the inclusion and exclusion criteria, if the subject will continue with the remaining study procedures. Subjects already on antipsychotics or other medications will continue use of the medications while participating in the current study. Subjects will not be taken off their antipsychotic medications for participation in this study.

Eligible subjects (acutely agitated subjects with schizophrenia, schizoaffective, or schizophreniform disorder) may be identified in out-patient clinics, mental health, psychiatric or medical emergency services, including medical/psychiatric observation units, or as newly admitted to a hospital setting for acute agitation or already in hospital for chronic underlying conditions. Subjects may be domiciled in our clinical research setting or hospitalized while undergoing screening procedures to assess eligibility.

### Example Statistical Considerations:

Outcomes can be summarized descriptively and assessed for normality prior to analysis using normal probability plots and Kolmogorov test statistics. Transformations or non-parametric analyses will be performed as necessary. All tests will be two-sided and considered statistically significant at alpha=0.05. Post-hoc comparisons will be performed as appropriate and significance levels for secondary analyses will be adjusted for multiple tests using the Bonferroni correction. Analyses can be performed using SAS, version 9.3 (SAS Institute Inc., Cary, N.C.). Linear mixed models can be used assess symptom improvement as measured by the PANSS-EC and RASS.

Descriptive statistics at each visit and the changes from baseline for clinical laboratory analyte values can be summarized by treatment cohort. Laboratory data may also be summarized by presenting shift tables using normal ranges, summary statistics of raw data and change from baseline values (means, medians, standard deviations, ranges) and by flagging notable values in data listings. Descriptive statistics and the changes from baseline for vital sign measurements can be summarized.

### Example Populations for Analysis:

Safety analyses can be based on the safety population that can include randomized participants who ingested at least 1 dose of double-blind study drug. Pharmacokinetic data analyses can be based on the intent-to-treat population that will include randomized participants who ingested at least 1 dose of double-blind study drug (dexmedetomidine hydrochloride) and have post-baseline PK assessments performed.

### Example Pharmacokinetic Analysis:

The following PK parameters for study drug (dexmedetomidine hydrochloride) can be calculated or derived from the data:

The concentration at 30-minute post-dose

The concentration at the time that the endpoint of temporarily arousable sedation by verbal stimulation is achieved.

### Example Pharmacodynamic Analysis:

Efficacy: Achievement of temporarily arousable sedation by verbal stimulation (dose and time to obtainment, duration once dosing stopped). PANS S-EC and ACES can be the primary measure. Descriptive analysis of dose needed to achieve an ACES of 5-7 in the shortest time without causing blood pressure or heart rate changes below the acceptable safety thresholds, as established by the protocol.

Repeated measures: ANOVAs can then be calculated, and effect sized reported (Cohen's d and np2, in %), using alpha level of 0.05 to determine statistical significance. Intertrial differences in cortisol, average heart rate, blood pressure, and salivary amylase will be calculated in a similar fashion.

### Example 3

A feasibility study to evaluate passive collection of activity data in subjects with agitation in the context of delirium or dementia.

TABLE 4

| | |
|---|---|
| Primary Objective | Primary Endpoints |
| 1. Evaluate the feasibility of passively | 1. The feasibility of passive and continuous data |
| collecting motion related, physiological | collection was determined by total time and |
| and audio data with mobile devices | percentage of continuous data collection for |
| (iPhone, Apple Watch) running custom | each stream of data aiming for >50% |
| software. | coverage. |
| Secondary Objective | Secondary Endpoints |
| 1. Determine the tolerability of carrying a | 1. The secondary endpoint was measured by |
| smartphone and wearing a data | Caregiver and Staff engagement with the |
| collection sensor on the wrist and/or | eCOA and EMA (threshold 80% |
| hand in a population of subjects who | completion) and responses to usability |
| may have frequent episodes of agitation | questionnaires at week 1 and 4 to provide |
| or impulsive behavior. | feedback on comfort, usability and |
| | engagement. |
| Exploratory Objectives | Exploratory Endpoints |
| 1. Evaluate the suitability of individual data | 1. The exploratory endpoint was measured by |
| streams and their combinations for purposes | comparison of data collected from the |
| of identification of agitation episodes in | smartphone and wearable device to episodes |
| passively collected data. | identified by subject or caregiver |
| | assessment: |
| 2. Determine how the smartphone, wrist or body | a. Cleaned single channel data |
| worn sensors, and applications affect subject | compared to assessments |
| interactions with Caregivers, HCP, and | b. Cleaned multichannel data |
| research staff. | compared to assessments |
| | c. Analyzed multichannel data |
| | compared to assessments |
| | d. Subject/Caregiver assessment |
| | data compared to agitation scale |
| | ratings |
| | e. Agitation scale ratings compared |
| | to cleaned single and multichannel |
| | data and analyzed multichannel |
| | data. |
| | f. Merged subject/caregiver |
| | assessment and multichannel data |
| | compared to agitation scale ratings |
| | 2. Caregiver and HCP questionnaires and |
| | interviews. |

### Example Study Design and Plan:

This was a multi-center, observational, feasibility study, to evaluate long term passive data collection, data quality, and user experience of an application to collect motion, location, physiological, and audio data with mobile devices (iPhone, Apple Watch).

The purpose of this study was to evaluate and improve data collection and usability in subjects experiencing agitation in the context of delirium or dementia.

Subjects with delirium and dementia were enrolled on separate cohorts. For subjects living at home their primary caregiver provided feedback on episodes of agitation. For subjects residing in a facility, HCP, and research staff provided feedback on episodes of agitation by completing the daily agitation form, including the PAS, for example, once per day. In some instances, passive data was not collected from caregivers. Subjects residing in a family home, group home, nursing home, assisted living, or specialty residential facilities including hospitals, geriatric psychiatry or other residential psychiatry units were eligible to participate. The dementia cohort opened first.

In some instances, all individuals who met eligibility criteria were enrolled.

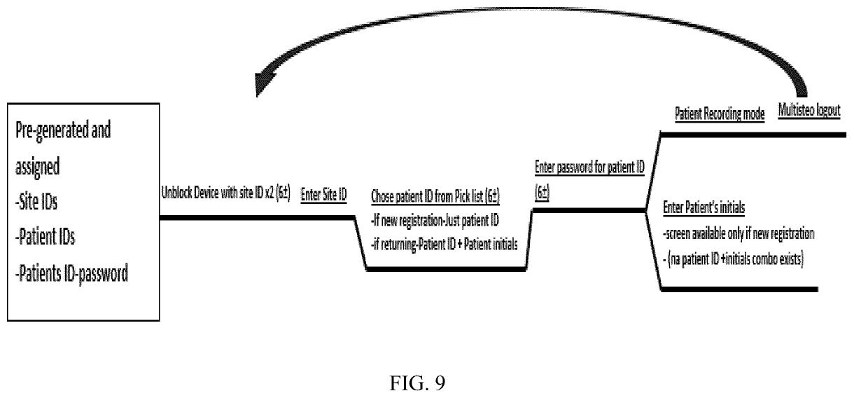

FIG. 9 illustrates a system flow diagram of a process to assign Patient IDs, Patient registration and recording of the data according to another embodiment of the present disclosure. User Flow description (see FIG. 9):
Dementia study:
Enrollment Flow
   Pre-generated & assigned:
   Site IDs
   Patient IDs
   Patient ID-password
      Staff & patient they have a mobile
      Lock is site ID ×2
      Single app mode runs
      Input site ID (maybe a standalone screen?)
      Select patient ID from pick list
      input patient initials
      Recording screen
      Settings button->logout option->site ID screen
   Patient
      Carries phone and wears a watch (or ring).
      Does not provide ePROs.
   Research Sit Staff
      manages subject devices
         sets up devices (watch & phone) on patient every morning,
         takes them off patient and puts them on a charging station every evening
         Checks for issues and is target for UX UI assessment
      provides EMA
         Responses provided after every visit of a patients, via dedicated device (tablet) and dedicated app:
         5 VAS for:
         Aberrant Vocalization
         Motor Agitation
         Aggressiveness
         Resisting Care
         Complications
   Clinician and selected staff

Enrolls patient to study

Is assigned ID

Manages patient ID & password list

Provides eCOA-PAS-assessment daily [rating period is 24 h] via dedicated device (tablet) and dedicated app

Off-boards patient(s) from study

In some instances, all subjects were issued an automated monitoring device (e.g., a waist worn multi-sensor device with networking capability such as iPhone; a wrist worn multi-sensor device with networking capability such as an AppleWatch; a finger worn multi-sensor device with networking capability such as Oura ring or the like) which run agitation monitoring apps.

Example Tech and Features can include: for a monitoring device similar to an Apple iPhone, the sensors & data types can include motion and location [e.g., time/date/duration tracking for any recording session]. In some instances, the raw data collection configuration [saving 0.8 MB/minute] can include an accelerometer (e.g., frequency—50 Hz), a gyroscope (e.g., frequency—50 Hz), and a compass (e.g., frequency—50 Hz). In some instances, if all tracked 3 GB data in 24 hours (rather demanding on traffic). The sensors and data types can also include audio data [e.g., time/date/duration tracking for any recording session] with a recording format of M4A: 16 khz sampling rate.

In some implementations, for a monitoring device similar to AppleWatch, the sensors and data types can include motion & location (e.g., time/date/duration tracking for any recording session). In some instances, the raw data collection configuration [saving 0.8 MB/minute] can include (1) location (latitude longitude and latitude) (e.g., GPS), precision—for, e.g., 14 decimal places, and frequency—Highest for device—approx. 1 record/second, (2) accelerometer (frequency—50 Hz), and (3) compass (frequency—50 Hz). In some instances, the sensors and data types can also include iOS pre-processed device motion data [saving 1.2 MB/minute] and Gyroscope (e.g., record every 50 Hz—with eliminated environment bias (e.g. gravity) If all tracked 3 GB data in 24 hours (rather demanding on traffic). In some instances, the physiological data can include: HR, Step count, Active energy, Basal energy, Stair claim, and/or the like.

In some implementations, for a monitoring device similar to the Oura ring, the Oura Cloud API can be a collection of HTTP REST API endpoints and uses OAuth2 for authentication. The sensors and data types can include (1) Pulse waveform and pulse amplitude variation detection with infrared PPG, (2) Body temperature, (3) 3D accelerometer and gyroscope, (4) Signals the Oura ring processes includes (e.g., Interbeat interval (IBI), Pulse amplitude variation (related to blood pressure variation), ECG level resting heart rate (RHR), Heart rate variability (HRV), Respiratory rate).

In some implementations, the recording protocol can include App record continuously until battery dies, App records from the moment you switch on the device & app on, App records while charging, after device restart (by user of b/c of low battery), app needs to trigger data collection manually. If battery under 20 percent—don't upload only recordings, for example.

In some implementations, the data upload protocol can include configurated for periodic saving of data [every 5 minutes], periodic sending of data [every 30 minutes], keep data backed on the device if until the batch is sent successfully-delete only after successful upload. For iPhone 8 or AppleWatch S3 to server upload done via WiFi & cellular data program, Optimised for wifi as the main upload channel. If wifi is not available for more then send via cellular.

The charging protocol can include overnight charging

The Login/ID: Caregiver inputs patient's ID & siteID & patient initials during the onboarding process; Patients are incapable of login on their own; Caregiver pairs watch with phone (in case of Applewatch S3)

In some implementations, alerts are sent to a server and are not visible for patients.

In some implementations, crash analytics & active monitoring can include data upload failed/device off, Phone static for more than 20 hours, Alert send if battery is lower than 20%.

In some implementations, the screens configuration can include device locked down—no access to other apps. App runs on background—no screen or (if screen required) black screen with status minimal screen. On Watch app, the screen has to be password protected

In some implementations additional technology can be added to the software suite or the devices: including apps to collect observer feedback. In some implementations, other sensors can be added for additional data collection (e.g. body temperature) or substituted for the automated monitoring device.

Study duration was four (4) weeks. Subjects wore the devices during waking hours for the duration of the study.

Types of data collected can include (1) Passive:
Location (latitude, longitude and altitude) (e.g., GPS)
Localisation (mobile signal stations & wifi)
Accelerometric data
Angular velocity (gyroscope)
Orientation (magnetometer/compass)
Number of steps (pedometer)
Activity type (time & confidence for activity type)
Audio data (for recognition of speech pace sentiment and impulsive movements)
Heart rate & heart rate variability

Types of data collected can include (2) Caregiver/Staff responses: Observer reports of agitation episodes and Usability questionnaires

At the end of their participation Caregivers or Staff returned the devices in a prepaid mailer.

Data was not monitored in real time during the course of the study. Participants were instructed to contact their physician for any changes in their health that they experienced during the study. Unanticipated problems with the Apps and devices were collected throughout the study.

Feasibility:

Feasibility was assessed based on the coverage of data collection and usability feedback from Caregivers, HCP and research staff. The threshold for passive data collection was the total time and percentage of continuous collection for each stream of data above 50% coverage. The target for tolerability was continuous wear of the iPhone, AppleWatch during daytime activities, every day. Gaps in wear were evident in the data and usability questionnaires provided feedback on challenges to hardware adherence.

In addition to the subject data, metrics for the devices' functionality was available from the operational cores of the devices, to understand battery life, app function at different battery levels, and any differences in app function under planned use versus pre-study testing.

### Example Study Populations

### Selection of Study Populations:

This study enrolled subjects with a diagnosis of delirium or dementia who experienced agitation severe enough to interfere with activities of daily living (ADLs) or social interaction. Subjects were identified in hospitals, skilled nursing facilities, nursing homes, or other residential care, and in outpatient practices. For enrolled subjects who were living at home, a caregiver provided feedback about subject's agitation episodes and managing subject's devices. This study enrolled up to 160 adult subjects at multiple sites in delirium or dementia cohorts. All participants were at least 18 years old on the day of consent. The dementia cohort opened first, enrolling up to 80 subjects with dementia.

Example Inclusion Criteria—Delirium
1. Male and female subjects 18 years and older.
2. Subjects who met DSM-5 criteria for delirium, measured by the Confusion assessment method (CAM) and the DRS-R-98.
3. Subjects with a recent history of agitation to a point that impaired social activities, requires staffing or medical intervention (kick, bite, flailing, etc.), impaired ability for functional activities of daily living, as disclosed by a caregiver or documented in the medical record.
4. Subjects residing in a family home, group home, nursing home, or assisted living were eligible to participate.
5. Subjects who could read, understand and provide written informed consent or who had a Legally Acceptable Representative (LAR)
6. Subjects who were willing and able to carry a smartphone and wear an activity tracker on their wrist or hand, alone or with the help of a caregiver.
7. Subjects who, either alone or with a caregiver, were able to operate a smartphone and wrist or hand-worn activity tracker, alone or with the help of a caregiver.
8. Subjects who were in good general health prior to study participation as determined by a detailed medical history, and in the opinion of the Principal Investigator.
9. Subjects, who were able to ambulate without an assistive device, or with a single point cane.

Example Exclusion Criteria—Delirium
1. Subjects hospitalized in an intensive care unit
2. Subjects experiencing delirium in the aftermath of stroke, major cardiac event, sepsis, or a hypoxic event
3. Subjects experiencing delirium as a result of polypharmacy.
4. Subjects who were unwilling or unable to carry or have a smartphone in their room, and wear an activity tracker on their wrist or body.
5. Subjects with serious or unstable medical illnesses. These included current hepatic (moderate-severe hepatic impairment), renal, gastroenterological, respiratory, cardiovascular (including ischemic heart disease, congestive heart failure), endocrinologic, neurologic or hematologic disease.
6. Subjects who were considered by the investigator, for any reason, to be an unsuitable candidate.

Example Inclusion Criteria—Dementia
1. Male and female subjects 18 years and older.
2. Subjects who met DSM-5 criteria for Dementia (all cause)
3. Subjects with a recent history of agitation in the past 6 months to a point that impaired social activities, required staffing or medical intervention (kick, bite, flailing, etc.), impaired ability for functional activities of daily living, as disclosed by a caregiver or documented in the medical record.
4. Subjects residing in a family home, group home, nursing home, or assisted living are eligible to participate.
5. Subjects who could read, understand and provided written informed consent or who have a Legally Acceptable Representative (LAR)
6. Subjects who were willing and able to carry a smartphone and wear an activity tracker on their wrist or hand, alone or with the help of a caregiver.
7. Subjects who, either alone or with a caregiver, were able to operate a smartphone and wrist or hand-worn activity tracker, alone or with the help of a caregiver.
8. Subjects who were in good general health prior to study participation as determined by a detailed medical history, and in the opinion of the Principal Investigator.
9. Subjects, who were able to ambulate without an assistive device, or with a single point cane.

Example Exclusion Criteria—Dementia

1. Subjects who were unwilling or unable to carry a smartphone and wear an activity tracker on their wrist or hand.
2. Subjects with serious or unstable medical illnesses. These included current hepatic (moderate-severe hepatic impairment), renal, gastroenterological, respiratory, cardiovascular (including ischemic heart disease, congestive heart failure), endocrinologic, neurologic or hematologic disease.
3. Subjects who were considered by the investigator, for any reason, to be an unsuitable candidate.

### Schedule of Events

TABLE 5
Schedule of Events, Residential Facility

| | | Daily | Week 1 | Week 4 |
|---|---|---|---|---|
| | Screening/ | (BL to | (+3 | (+3 |
| Activity | Baseline | EOS) | days) | days) |
| Informed consent | X | | | |
| Inclusion/Exclusion criteria | X | | | |
| Demographics | X | | | |
| Medical History¹ & Medications | X | | X | X |
| Mini Mental State Exam | X | | | |
| Agitation History | X | | | |
| Device accountability | X | | | |
| Device training (subject) | X | | | |
| Unanticipated problems/ADEs | X | | X | X |
| Observer agitation form¹ | | X | | |
| Passive data collection | | X | | |
| Device return² | | | | X |
| Usability questionnaire³ | | | (X) | (X) |

TABLE 6
Schedule of Events, Outpatient

| | Screening/ | Daily | Week 1 | Week 4 | Unsched |
|---|---|---|---|---|---|
| Activity | Baseline | (BL to EOS) | (+3 days) | (+3 days) | Call |
| Informed consent | X | | | | |
| Inclusion/Exclusion criteria | X | | | | |
| Demographics | X | | | | |
| Medical History¹ & Medications | X | | X | X | |
| Mini Mental State Exam | X | | | | |
| Agitation History | X | | | | |
| Device accountability | X | | | | |
| Device training (Caregiver and | X | | | | |
| subject) | | | | | |
| Unanticipated problems/ADEs | X | | X | X | (X) |
| Observer agitation form^{1,5} | | X | | | |
| Passive data collection | | X | | | |
| Compliance call | | | X | | |
| End of study call² | | | | X | |
| Unscheduled call⁴ | | | | | |
| Device return² | | | | X | |
| Usability questionnaires³ | | | X | X | |

TABLE 7
Schedule of Events, Decentralized⁶

| Activity | | | | | | | |
|---|---|---|---|---|---|---|---|
| (all | | | Daily | | Week 1 | Week 4 | |
| conducted | Screening/ | | (BL to | | (+3 | (+3 | Unsch |
| remotely) | Baseline⁶ | Training⁶ | EOS) | Weekly | days) | days) | Call |
| Informed | X | | | | | | |
| consent | | | | | | | |
| Inclusion/Exclusion | X | | | | | | |
| criteria | | | | | | | |
| Demographics | X | | | | | | |
| Medical | X | | | | X | X | |
| History¹ & | | | | | | | |
| Medications | | | | | | | |
| Mini Mental | X | | | | | | |
| State Exam | | | | | | | |
| Agitation | X | | | | | | |
| History | | | | | | | |
| Ship devices to | X | | | | | | |
| subject | | | | | | | |
| Device | X | X | | | | | |
| accountability | | | | | | | |
| Device | | X | | | | | |
| training | | | | | | | |
| (Caregiver | | | | | | | |
| and subject) | | | | | | | |
| Unanticipated | | X | | | X | X | (X) |
| problems/ADEs | | | | | | | |
| Observer | | | X | | | | |
| agitation form^{1,5} | | | | | | | |
| Passive data | | | X | | | | |
| collection | | | | | | | |
| Compliance | | | | X | | | |
| emails/texts | | | | | | | |
| Compliance call | | | | | X | | |
| End of study call | | | | | | X | |
| Unscheduled | | | | | | | X |
| call⁴ | | | | | | | |
| Device return² | | | | | | X | (X) |
| Usability | | | | | (X) | (X) | |
| questionnaires³ | | | | | | | |

¹Validated, condition-specific tools will be used in each cohort to assess the eligible diagnosis and agitation.
²Sites will collect devices from subjects and return to Sponsor. For outpatient and virtual subjects they will return devices to the site. Site will return them to Sponsor.
³A usability questionnaire will be administered at least once during the study.
⁴If a subject's devices are not transmitting data for more than 24 hours, Sponsor may ask the site to reach out to the participant and troubleshoot. Unscheduled calls should only be prompted by the Sponsor.
⁵The observer agitation form will be completed by research staff in a residential setting and by a caregiver in the outpatient and virtual settings.
⁶When the study is run decentralized there are no in-person visits. Screening/Baseline and Training visits should utilize teleconference tools so the subject, caregiver, and study team can see and speak to each other.

Example Cohort Size

This study enrolled up to 160 adult subjects at multiple sites in delirium or dementia cohorts. The total number of participants for each diagnosis were enrolled in smaller cohorts of 5, 10 or 20. The maximum size for each cohort was 80 participants.

Example Decentralized Dementia Cohort

This study included a decentralized cohort of up 30 subjects. This cohort included only dementia patients who were residing at home with their primary caregiver.

Example Recruitment

Subjects were recruited by HCP referral, via online advertising, and at participating hospitals, clinics or specialty facilities for each of the targeted diagnoses. Caregivers were asked by HCP or research staff to provide feedback when subjects were living at home. All recruitment material was submitted for IRB approval.

### Example Study Procedures

### Preparing Devices

Study devices were shipped to the site for distribution to study participants, or directly to the caregiver. Upon receipt research staff prepared the devices as follows:
Compared shipping inventory with devices received
Plugged in devices to fully charge
Completed set-up of devices using the Study Device Manuals.

Caregivers assisted subjects in the decentralized cohort participated in a training session after they received the devices.

When the devices were fully charged and the Apps were downloaded, they were powered off and stored.

Screening/Baseline

Subjects were screened and met eligibility criteria before data collection began.

If subjects completed the study without an in-person visit Screening/Baseline took place over two sessions. One to complete consent and all eligibility assessments and one for training after the caregiver received devices from the site

The following procedures were performed at Screening/Baseline.
Obtained written informed consent from subject or LAR
Provided Caregiver with information sheet
Reviewed Inclusion and Exclusion criteria
Collected demographic information
Recorded medical history, including prior and current therapies (e.g. prescription and nonprescription medications)
Administered Mini Mental State Exam (MMSE)
Confirmed recent history of agitation severe enough to interfere with ADLs or social interactions
Device accountability
Demonstrated and trained caregivers and subjects on operation, charging, and return of devices; and use of Apps.
Documented any Unanticipated Problems/Adverse Device Events

Daily (Baseline through end of study 28 (+3) days)
Caregivers or facility staff assisted subjects with putting on Apple Watch iPhone
Subjects wore Apple Watch during waking hours
Subjects carried iPhone during waking hours
Caregivers or research staff completed the PAS once per day
Caregivers or research staff set Apple Watch, iPhone to charge overnight

End of Week 1 (+3 days)
Caregivers or research staff completed usability questionnaire

Research staff called caregivers:
Reminder about usability questionnaire
Asked about any issues with adherence
Documented any Unanticipated Problems/Adverse Device Events

End of Study (Day 22 (+5 days))
Caregivers or research staff completed usability questionnaire
Research staff called caregivers:
   Reminder about usability questionnaire
   Asked about any issues with adherence
   Documented any Unanticipated Problems/Adverse Device Events
   Reminder to power off and return devices, answer any questions about the return process

Additional Study Communication

Texts/Emails

For the Decentralized Dementia Cohort, communications with the caregiver to support adherence, notification or follow-up of technology issues occurred per the caregivers preferred route, and occurred up to weekly.

Unscheduled Calls

For the Outpatient and Decentralized cohort, in the event that data from a subject did not reach the servers in more than 24 hours Sponsor might ask the site to reach out to the caregiver to inquire about issues with the devices or changes to subject participation.

Return of Devices

Outpatient/Decentralized Caregivers were provided with addressed, prepaid shippers to return the study devices. Participants returned the devices at the end of their active study period.

At sites where patients were residents, research staff returned the devices in the addressed, prepaid shippers provided by Health Mode. The return process included:
Document each device to be returned on the device accountability page of the EDC
Power off all devices
Pack and ship devices with supplied material.

Study assessments

Confusion Assessment Method (CAM)

The Confusion Assessment Method is a diagnostic tool for identifying delirium and distinguishing it from other types of cognitive impairment. The CAM is valid when administered by non-psychiatrist, clinical raters. Answers to nine questions inform the presence or absence of four features of 3 of which must be present to confirm a diagnosis of delirium.

Delirium Rating Scale-Revised (DRS-R-98)

The Delirium Rating Scale-Revised is the 1998 revision of the Delirium Rating Scale (1988) to include items which improve its use as a diagnostic tool. For the purposes of this study, the desirable feature of the DRS-R-98 is its power and validity as a repeatable measure of severity of delirium. The DRS-R-98 can be administered by any trained clinician.

Pittsburgh Agitation Scale (PAS)

The Pittsburgh Agitation Scale (PAS) is an instrument based on direct observations of the subject, developed to monitor the severity of agitation associated with dementia. Four domains —Aberrant Vocalization, Motor Agitation, Aggressiveness, Resisting Care—are rated from 0-4 to give a sense of the subject's most severe agitation in a defined period of observation.

Mini Mental State Exam (MMSE)

The Mini Mental State Exam is an instrument based on interview with the subject to assess cognitive function in multiple domains: registration, attention and calculation, recall, language, ability to follow simple commands and orientation. It is used as a screen for dementia and to assess severity of cognitive impairment. The exam is scored out of 30 points with lower scores indicating more severe impairment.

Safety

Unanticipated Problems

Definition of Unanticipated Problems (UP)

The Office for Human Research Protections (OHRP) considered unanticipated problems involving risks to participants or others to include, in general, any incident, experience, or outcome that meets all of the following criteria:
Unexpected in terms of nature, severity, or frequency given (a) the research procedures that are described in the protocol-related documents, such as the Institutional Review Board (IRB)-approved research protocol and Informed Consent document; and (b) the characteristics of the participant population being studied;
Related or possibly related to participation in the research (“possibly related” means there is a reasonable possibility that the incident, experience, or outcome may have been caused by the procedures involved in the research); and
Suggests that the research places participants or others at a greater risk of harm (including physical, psychological, economic, or social harm) than was previously known or recognized.

This definition could include an unanticipated adverse device effect, any serious adverse effects on health or safety or any life-threatening problem or death caused by, or associated with, a device, if that effect, problem, or death was not previously identified in nature, severity, or degree of incidence in the investigational plan or application (including a supplementary plan or application), or any other unanticipated serious problem associated with a device that relates to the rights, safety, or welfare of subjects (21 CFR 812.3(s)).

Unanticipated Problem Reporting

The principal investigator (PI) reported unanticipated problems (UPs) to the selected commercial Institutional Review Board (IRB) and to the sponsor. The UP report might include the following information:
Report date, IRB Study number, Study Title, Study Staff Contact Information, Date UP occurred, and date PI was notified about the UP.
Description of the Unanticipated Problem which occurred during the conduct of the research.
Provide an explanation for why this Unanticipated Problem occurred.
Characterize the impact of the Unanticipated Problem on the study.
Describe the steps which have been taken to resolve the reported occurrence.
Describe the plan implemented to avoid or prevent future occurrences.
Inform other study participants as necessary.
Name all other entities to which this UP has been reported.
Determine if the UP will require modification of the currently approved study and/or consent form.

Serious Adverse Event (SAE) Reporting

Adverse events and deaths occurring in the course of an approved study that were serious, unanticipated and related or probably related to use of the apps or the devices, by the judgment of the investigator, were reported to the IRB.

In some instances, if the event satisfies all three of these criteria the event was reported to the IRB within 5 business days of learning of the event. The study sponsor was also notified within 24 hours of the site learning of the event.

Statistical Methods

Statistical Analyses

A statistical analysis plan (SAP) that described the details of the analyses to be conducted was finalized before database lock.

Continuous variables were summarized by treatment using descriptive statistics (n, mean, median, standard deviation, minimum, and maximum). For categorical variables, frequencies and percentages were presented by data type. Baseline was defined as the last observation prior to initiation of study data collection. Details of the statistical analyses were provided in the Statistical Analysis Plan, which was finalized prior to database lock.

Feasibility Analysis

The data of all subjects enrolled was evaluated to measure feasibility. Subjects were stratified by percentage of data collected and group characteristics were examined for trends and opportunities to optimize data collection coverage.

### Example Data Handling

### Example Data Extract, Transform and Load (ETL) Processes

The data extract, transform, and load (ETL) process is depicted in FIG. 2. A software program was used to extract data from various internal or external sensors of the mobile device. The software application included a reporting system used to track any issues with usage, data collection and transfer. Data processing steps were incorporated in various stages of the ETL process. Data processing steps included file compression, encryption, timestamping, elimination of silence, speech masking or preliminary speech analysis. Last steps in processing included data analytics providing outcome measures to support primary endpoint; and advanced agitation and hyperirritability characteristics providing outcome measures to support exploratory endpoints.

Study Discontinuation and Closure

This study might be temporarily suspended or prematurely terminated if there was sufficient reasonable cause. Written notification, documenting the reason for study suspension or termination, was to be provided by the suspending or terminating party to study participants, investigator, sponsor and regulatory authorities. If the study was prematurely terminated or suspended, the Principal Investigator (PI) promptly informed study participants, the Institutional Review Board (IRB), and sponsor and provided the reason(s) for the termination or suspension. Study participants were contacted via phone or email and be informed of changes to study schedule.

Circumstances that might warrant termination or suspension included, but were not limited to:
Determination of unexpected, significant, or unacceptable risk to participants
Demonstration of efficacy that would warrant stopping
Insufficient compliance to protocol requirements
Data that were not sufficiently complete and/or evaluable
Determination that the primary endpoint had been met
Determination of futility

Study might resume once concerns about safety, protocol compliance, and data quality were addressed, and satisfied the sponsor, IRB and/or Food and Drug Administration (FDA).

Withdrawal

If a participant was withdrawn from this study, the reason(s) for withdrawal was reported to the study data collection system. Data collected up to the point of withdrawal was used for analysis and retained per protocol. No further user interaction data was collected from the participant following their withdrawal.

Although the disclosure herein has been described with reference to particular embodiments, it is to be understood that these embodiments are merely illustrative of the principles and applications of the present disclosure. Many modifications and variations will be apparent to those skilled in the art. The embodiments have been selected and described in order to best explain the disclosure and its practical implementations/applications, thereby enabling persons skilled in the art to understand the disclosure for various embodiments and with the various changes as are suited to the particular use contemplated. It is therefore to be understood that numerous modifications may be made to the illustrative embodiments and that other arrangements may be devised without departing from the spirit and scope of the present disclosure as defined by the appended claims.

The illustrations of overview of the system as described herein are intended to provide a general understanding of the structure of various embodiments, and they are not intended to serve as a complete description of all the elements and features of apparatus and systems that might make use of the structures described herein. Many other arrangements will be apparent to those skilled in the art upon reviewing the above description. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific figures have been illustrated and described herein, it should be appreciated that any other designs calculated to achieve the same purpose may be substituted for the specific arrangement shown. This disclosure is intended to cover any and all adaptations or variations of various embodiments of the present disclosure. Combinations of the above designs/structural modifications not specifically described herein, will be apparent to those skilled in the art upon reviewing the above description. Therefore, it is intended that the disclosure not be limited to the particular method flow, apparatus, system disclosed as the best mode contemplated for carrying out this disclosure, but that the disclosure will include all embodiments and arrangements falling within the scope of the appended claims.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

While various embodiments have been described above, it should be understood that they have been presented by way of example only, not limitation, and various changes in form and details may be made. Any portion of the apparatus and/or methods described herein may be combined in any combination, except mutually exclusive combinations. The embodiments described herein can include various combinations and/or sub-combinations of the functions, components and/or features of the different embodiments described.

1. A method, comprising:
receiving first physiological data of sympathetic nervous system activity;
establishing a baseline value of at least one physiological parameter by training at least one machine learning model using the first physiological data;
receiving, from a first monitoring device attached to a subject, second physiological data of sympathetic nervous system activity in the subject;
analyzing, using the at least one machine learning model and based on the baseline value of at least one physiological parameter, the second physiological data to predict an agitation episode of the subject; and
sending, based on predicting the agitation episode of the subject, a signal to a second monitoring device to notify the second monitoring device of the prediction of the agitation episode of the subject such that treatment can be provided to the subject to decrease sympathetic nervous system activity in the subject.

2. The method of claim 1, wherein: the first monitoring device is a wearable device in contact with the subject.

3. The method of claim 1, wherein the second monitoring device is monitored by a caregiver of the subject.

4. The method of claim 1, wherein: the analyzing to predict the agitation episode includes determining a time period within which the agitation episode of the subject will occur.

5. The method of claim 1, wherein:
the analyzing to predict the agitation episode includes determining a degree of the agitation episode of the subject.

6. The method of claim 1, wherein:
the analyzing to predict the agitation episode includes:
comparing the second physiological data with the baseline value of at least one physiological parameter;
when the second physiological data exceeds a first threshold of the baseline value, the signal is a first signal, the treatments are first treatments;
when the second physiological data exceeds a second threshold of the baseline value, the signal is a second signal different from the first signal, the treatments are second treatments different from the first treatments.

7. The method of claim 1, wherein the receiving the second physiological data is during a first time period; the method further comprises:
receiving, during a second time period after the first time period, third physiological data of sympathetic nervous system activity in the subject; and
generating, based on the second physiological data and the third physiological data, a report of sympathetic nervous system activity in the subject to identify a pattern of a change of sympathetic nervous system activity in the subject.

8. The method of claim 1, wherein:
the treatment includes administering an anti-agitation agent to the subject.

9. The method of claim 1, wherein:
the second physiological data of sympathetic nervous system activity include at least one of a change in electrodermal activity, heart rate variability, cognitive assessments such as pupil size, secretion of salivary amylase, blood pressure, pulse rate, respiratory rate, or level of oxygen in blood.

10. The method of claim 1, wherein:
the sympathetic nervous system activity is assessed by measuring any change in electrodermal activity or any change in electrodermal activity together with any change in resting electroencephalography.

11. The method of claim 1, further comprising:
receiving an indication associated with the agitation episode after sending the signal to the second monitoring device; and
further training the at least one machine learning model based on the indication.

12. The method of claim 1, further comprising:
receiving an indication associated with the agitation episode after sending the signal to the second monitoring device, the indication indicating at least one of (1) whether or not the agitation episode occurs, (2) when the agitation episode occurs, (3) a degree of the agitation episode, (4) a time period for which the agitation episode lasts, or (5) a symptom of the agitation episode; and
further training the at least one machine learning model based on the indication.

13. The method of claim 1, wherein:
the at least one machine learning model includes at least one of a linear regression, logistic regression, a decision tree, a random forest, a neural network, a deep neural network, or a gradient boosting model.

14. The method of claim 1, wherein:
the at least one machine learning model is trained based on at least one of supervised learning, unsupervised learning, semi-supervised learning, or reinforcement learning.

15. The method of claim 1, wherein:
the analyzing to predict the agitation episode includes determining, based on a comparison between the second physiological data and the baseline value, a degree of the agitation episode of the subject.

16. The method of claim 1, further comprising:
receiving, from the first monitoring device, additional data of sympathetic nervous system activity in the subject, the additional data including at least one of audio data, motion data, or location data,
the analyzing includes analyzing, using the at least one machine learning model, the additional data to predict the agitation episode of the subject.

17. An apparatus, comprising:
a memory; and
a processor operatively coupled to the memory, the processor configured to:
receive, from a first monitoring device attached to a subject, physiological data of sympathetic nervous system activity in the subject;
analyze, using at least one machine learning model, the physiological data to detect an anomaly from a reference pattern of sympathetic nervous system activity to determine a probability of an occurrence of an agitation episode of the subject; and
send a signal to a second monitoring device to notify the second monitoring device of the probability of the occurrence of the agitation episode of the subject such that treatment can be provided to the subject to decrease sympathetic nervous system activity in the subject.

18. The apparatus of claim 17, wherein:
the processor is configured to:
receive an indication associated with the agitation episode after sending the signal to the second monitoring device; and
further train the at least one machine learning model based on the indication.

19. The apparatus of claim 17, wherein:
the processor is configured to:
receive an indication associated with the agitation episode after sending the signal to the second monitoring device, the indication indicating one of (1) whether or not the agitation episode occurs, (2) when the agitation episode occurs, (3) a degree of the agitation episode, (4) a time period for which the agitation episode lasts, or (5) a symptom of the agitation episode; and
further train the at least one machine learning model based on the indication.

20. A processor-readable non-transitory medium storing code representing instructions to be executed by a processor, the code comprising code to cause the processor to:
receive, from a first monitoring device attached to a subject, physiological data of sympathetic nervous system activity in the subject;
analyze, using at least one machine learning model, the physiological data to detect an anomaly from a reference pattern of sympathetic nervous system activity to determine a probability of an occurrence of an agitation episode of the subject; and
send a signal to a second monitoring device to notify the second monitoring device of the probability of the occurrence of the agitation episode of the subject such that treatment can be provided to the subject to decrease sympathetic nervous system activity in the subject.

21. The processor-readable non-transitory medium of claim 20, wherein the code comprises code to cause the processor to:
train, prior to analyzing using the at least one machine learning model, the at least one machine learning model based on training physiological data of sympathetic nervous system activity associated with a plurality of subjects, the at least one machine learning model including a plurality of physiological parameters as input, each physiological parameter from the plurality of physiological parameters associated with a weight from a plurality of weights of the machine learning model;
determine, based on the at least one machine learning model, the reference pattern of at least one physiological parameter from the plurality of physiological parameters.

22. The processor-readable non-transitory medium of claim 20, wherein the code comprises code to cause the processor to:
train, prior to analyzing using the at least one machine learning model, the at least one machine learning algorithm based on training physiological data of sympathetic nervous system activity associated with a plurality of subjects, the at least one machine learning model including a plurality of physiological parameters as input, each physiological parameter from the plurality of physiological parameters associated with a weight from a plurality of weights of the machine learning models;
determine, based on the at least one machine learning model, the reference pattern of at least one physiological parameter from the plurality of physiological parameters, receive an indication associated with the agitation episode after sending the signal to the second monitoring device; and
further train, based on the indication, the at least one machine learning model to adjust the reference pattern of the at least one physiological parameter and a weight associated with the at least one physiological parameter.

23. A method of diagnosing an impending agitation episode in a subject predisposed to agitation comprising:
(a) monitoring one or more physiological signals of sympathetic nervous system activity in the subject using an automated sensoring device placed or mounted on the subject's skin surface; and
(b) identifying, via the processing of incoming data in the device, when the subject is about to have an agitation episode.

24. The method of claim 23, wherein the automated sensoring device is a wearable device.

25. The method of claim 23, wherein the physiological signals of sympathetic nervous system activity are selected from one or more of the following: change in electrodermal activity; heart rate variability (e.g. resting EEG, ECG); cognitive assessments such as pupil size; secretion of salivary amylase; blood pressure; pulse; respiratory rate; temperature variability and level of oxygen in the blood.

26. The method of claim 23, wherein sympathetic nervous system activity is assessed by measuring any change in electrodermal activity or any change in electrodermal activity together with any change in resting EEG.

27. The method of claim 23, wherein the automated sensoring device sends data of physiological signals related to sympathetic nervous system activity in the patient to a remotely situated apparatus (e.g. a computer database) that includes one or more early warning algorithm.

28. The method according to claim 27, wherein the device sends a signal to the remotely situated apparatus through Bluetooth.

29. The method of claim 23, wherein the subject is suffering from a neuropsychiatric disease selected from the group consisting of schizophrenia, bipolar disorder, bipolar mania, delirium, major depressive disorders and depression.

30. The method of claim 23, wherein the subject is suffering from a neurodegenerative disease selected from the group consisting of Alzheimer's disease, frontotemporal dementia (FTD), dementia, dementia with Lewy bodies (DLB), post-traumatic stress disorder, Parkinson's disease, vascular dementia, vascular cognitive impairment, Huntington's disease, multiple sclerosis, Creutzfeldt-Jakob disease, multiple system atrophy, traumatic brain injury and progressive supranuclear palsy.

31. The method of claim 23, wherein the subject is predisposed to agitation associated with opioid withdrawal, substance abuse withdrawal (including cocaine amphetamine), or alcohol withdrawal.

32. A method of alerting a caregiver to an impending agitation episode in a subject predisposed to agitation comprising:
(a) monitoring one or more physiological signals of sympathetic nervous system activity in the subject using an automated sensoring device placed or mounted on the subject's skin surface;
(b) identifying, via the processing of incoming data in the device, when the subject is about to have an agitation episode; and
(c) sending a signal from the device to a compatible device monitored by a caregiver alerting the caregiver to an impending agitation episode in the subject.

33. A method of preventing the emergence of agitation in a subject predisposed to agitation comprising:
(a) monitoring one or more physiological signals of sympathetic nervous system activity in the subject using an automated sensoring device placed or mounted on the subject's skin surface;
(b) identifying, via the processing of incoming data in the device, when the subject is about to have an agitation episode;
(c) sending a signal from the device to a remote compatible device monitored by a caregiver alerting the caregiver to an impending agitation episode in the subject; and
(d) administering by the caregiver an anti-agitation agent which decreases sympathetic nervous activity in said subject.

34. The method of claim 33, wherein agitation is prevented or treated without causing significant sedation.

35. The method of claim 33, wherein the anti-agitation agent is an alpha-2 adrenergic receptor agonist.

36. The method of claim 35, wherein the alpha-2 adrenergic receptor agonist is selected from the group consisting of clonidine, guanfacine, guanabenz, guanoxabenz, guanethidine, xylazine, tizanidine, medetomidine, dexmedetomidine, methyldopa, methylnorepinephrine, fadolmidine, iodoclonidine, apraclonidine, detomidine, lofexidine, amitraz, mivazerol, azepexol, talipexol, rilmenidine, naphazoline, oxymetazoline, xylometazoline, tetrahydrozoline, tramazoline, talipexole, romifidine, propylhexedrine, norfenefrine, octopamine, moxonidine, lidamidine, tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof.

37. The method of claim 35, wherein the alpha-2 adrenergic receptor agonist is dexmedetomidine or a pharmaceutically acceptable salt thereof.

38. The method of claim 37, wherein the dexmedetomidine or the pharmaceutically acceptable salt thereof is administered parenterally by intravenous injection.

39. The method of claim 37, wherein the dexmedetomidine or the pharmaceutically acceptable salt thereof is administered sublingually using a self-supporting, dissolvable film.

40. The method of claim 37, wherein the dexmedetomidine is administered as the hydrochloride salt.

41. The method of claim 40, wherein dexmedetomidine hydrochloride is administered at unit dose in the range of about 5 micrograms to about 250 micrograms, preferably about 5 micrograms to about 200 micrograms.

42. The method of claim 40, wherein dexmedetomidine hydrochloride is administered at unit dose of 180 micrograms.

43. A method of treating the early stage emergence of agitation or the signs of agitation in a subject predisposed to agitation comprising:
(a) monitoring one or more physiological signals of sympathetic nervous system activity in the subject using an automated sensoring device placed or mounted on the subject's skin surface;
(b) identifying, via the processing of incoming data in the device, when the subject is having an agitation episode;
(c) sending a signal from the device to a remote compatible device monitored by a caregiver alerting the caregiver to the start of agitation episode in the subject; and
(d) administering by the caregiver an anti-agitation agent which decreases sympathetic nervous activity in said subject.
